(19) United States
(12) Patent Application Publication
(10) Pub. No.: US 2012/0197401 A1
(43) Pub. Date: Aug. 2, 2012

(54) INTERBODY SPINAL IMPLANTS WITH MODULAR ADD-ON DEVICES

(75) Inventors: James Duncan, Hernando, MS (US); Anthony Melkent, Memphis, TN (US); William Armstrong, Memphis, TN (US)

(73) Assignee: WARSAW ORTHOPEDIC, INC., Warsaw, IN (US)

(21) Appl. No.: 13/015,165

(22) Filed: Jan. 27, 2011

(51) Int. Cl. A61F 2/44; B23P 11/00

(52) U.S. Cl. 623/17.16; 29/428

(57) ABSTRACT

An implant assembly for a spinal column includes at least an interbody element and an add-on element that is removably attachable to the add-on element to form an implant assembly. At least a portion of the add-on element is positionable in a space between vertebrae along with the interbody element to support the vertebrae.

### BACKGROUND

The present invention relates generally to interbody spinal implants with modular add-on devices, and to methods and instruments for implanting the interbody spinal implants.

Several devices and systems have been developed for stabilizing the spine and for facilitating fusion at various levels of the spine. The spinal anatomy including the bony structure of vertebral bodies, vascular structures, neural structures, musculature, and other vital tissue along the spinal column make it difficult to position an interbody implant in the disc space between adjacent vertebral bodies or to engage a plate between the adjacent vertebrae. In addition, when an implant is placed into a disc space, the channel or path that the implant took to enter the disc space provides a path for retrograde movement of the implant from the disc space. The variability in the spinal anatomy and in the location of the trailing end of the implant relative to the adjacent vertebral bodies can make attaching a plate to the implant and/or to the adjacent vertebral bodies difficult to achieve.

As a result, additional improvements in spinal interbody fusion implants and insertion instruments and techniques are needed that provide the surgeon intraoperative flexibility to adapt the implant and implantation technique to fit patient needs.

### SUMMARY

According to one aspect, an interbody spinal implant assembly for a spinal column is disclosed that includes an interbody element positionable in a space between vertebrae and an add-on element that is secured to the trailing end of the interbody element to provide the implant assembly. The add-on element can be configured to reside entirely or substantially in the space between the vertebrae. The add-on element can also be configured to include one or more apertures to receive a bone anchor to secure the implant assembly to one or more of the vertebrae. In some embodiments, the add-on element includes at least one projection with the at least one aperture and the projection is configured to reside in or substantially in the space between the vertebrae. In other embodiments, the add-on element includes at least one projection that is configured to extend from the space between vertebrae and along an exterior surface of one or more of the vertebrae. In still other embodiments, the add-on element includes at least one projection that is configured to extend from the space between vertebrae and into a notch formed in the vertebra so that the projection is entirely or substantially recessed relative to the exterior face of the vertebra to form a no profile or low profile arrangement.

According to another aspect, there is disclosed an interbody implant assembly and method for assembling an interbody spinal implant that includes an add-on element removably attachable to an interbody element. The add-on element and interbody element include an interfacing mating portion and receiving portion that secures the add-on element to the trailing end of the interbody element. An inserter can be provided that engages the assembled interbody element and add-on element to facilitate insertion of the implant assembly into the space between vertebrae. The inserter can include a body with a guide providing passages aligned with apertures of the add-on element to facilitate formation of holes in the vertebrae to receive bone anchors and/or to guide the bone anchors into the vertebrae along a desired trajectory. Also described are reamer guides that include guide bodies to guide reamers in forming notches in the vertebrae that receive projections extending from the add-on element when a low or no profile arrangement is desired.

Related features, aspects, embodiments, objects and advantages of the present invention will be apparent from the following description.

### BRIEF DESCRIPTION OF THE DRAWINGS

FIG. 1 is a perspective view of one embodiment interbody element of a spinal implant assembly.

FIG. 2 is a perspective view of one embodiment add-on element attachable to the interbody element of FIG. 1.

FIG. 3 is an enlarged perspective view of connection portion of the interbody element of FIG. 1.

FIG. 4 is an enlarged perspective portion of a mating portion of the add-on element of FIG. 2.

FIGS. 5A-5C are perspective views showing connection of the add-on element to the interbody element of FIG. 1.

FIGS. 6A-6D are various views showing the add-on element of FIG. 2 attached to the interbody element of FIG. 1 in a spinal column segment.

FIG. 7 is a perspective view of another embodiment add-on element attachable to the interbody element of FIG. 1.

FIGS. 8A-8D are various views showing the add-on element of FIG. 6 attached to the interbody element of FIG. 1 in a spinal column segment.

FIGS. 9A-9D are various views of a method and instrument for preparing the vertebrae to receive the add-on element and interbody element of FIGS. 8A-8D.

FIGS. 10A-10D are various views of an inserter instrument and method for inserting the interbody element and add-on element of FIGS. 8A-8D and with the inserter including a guide to guide bone anchors through apertures of the add-on element.

FIGS. 11A-11D are various views showing another embodiment add-on element attached to the interbody element of FIG. 1 in a spinal column segment.

FIGS. 12A-12F are various views of a spinal column segment showing a method and instrument for preparing the vertebrae to receive the add-on element and interbody element of FIGS. 11A-11D in a low or no profile arrangement.

FIGS. 13A-13D are various views showing another embodiment add-on element attached to the interbody element of FIG. 1 in a spinal column segment.

FIGS. 14A-14C are various views of another embodiment inserter instrument and method for inserting the interbody element and add-on element of FIGS. 13A-13D and with the inserter including a guide to guide bone anchors through apertures of the add-on element.

FIGS. 15A-15D are various views showing another embodiment add-on element attached to the interbody element of FIG. 1 in a spinal column segment.

FIGS. 16A-16D are various views showing another embodiment add-on element attached to the interbody element of FIG. 1 in a spinal column segment.

FIGS. 17A-17D are various views showing another embodiment add-on element attached to the interbody element of FIG. 1 in a spinal column segment.

FIG. 18 is a perspective view of another embodiment interbody element attachable to the add-on elements disclosed herein.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices, and such further applications of the principles of the invention as illustrated herein are contemplated as would normally occur to one skilled in the art to which the invention relates.

Methods, techniques, instrumentation and implants are provided to restore and/or maintain a collapsed, partially collapsed, damaged, diseased, or otherwise impaired spinal disc space at a desired disc space height and adjacent endplate orientation. Methods, techniques, instrumentation and implants are provided to restore and/or maintain a space created by one or more removed vertebrae and associated discs. Access to the implantation site can be uni-portal, bi-portal, or multi-portal. The instruments and implants may also be employed in an anterior approach to the implantation site, although other approaches are also contemplated, including antero-lateral, postero-lateral, oblique, posterior, and lateral approaches. Also, the surgical methods, techniques, instruments and implants may find application at all vertebral segments of the spine, including the lumbar, thoracic and cervical spinal regions.

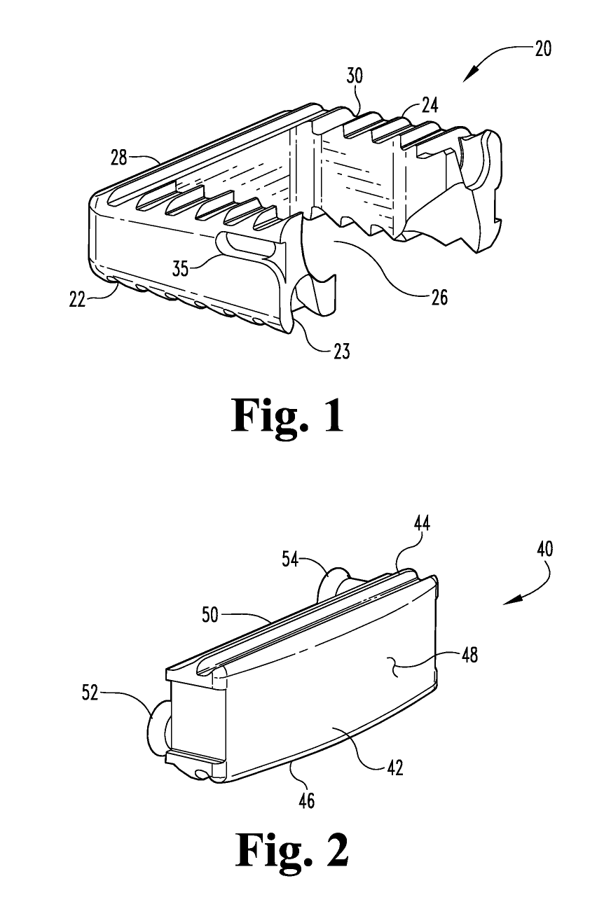

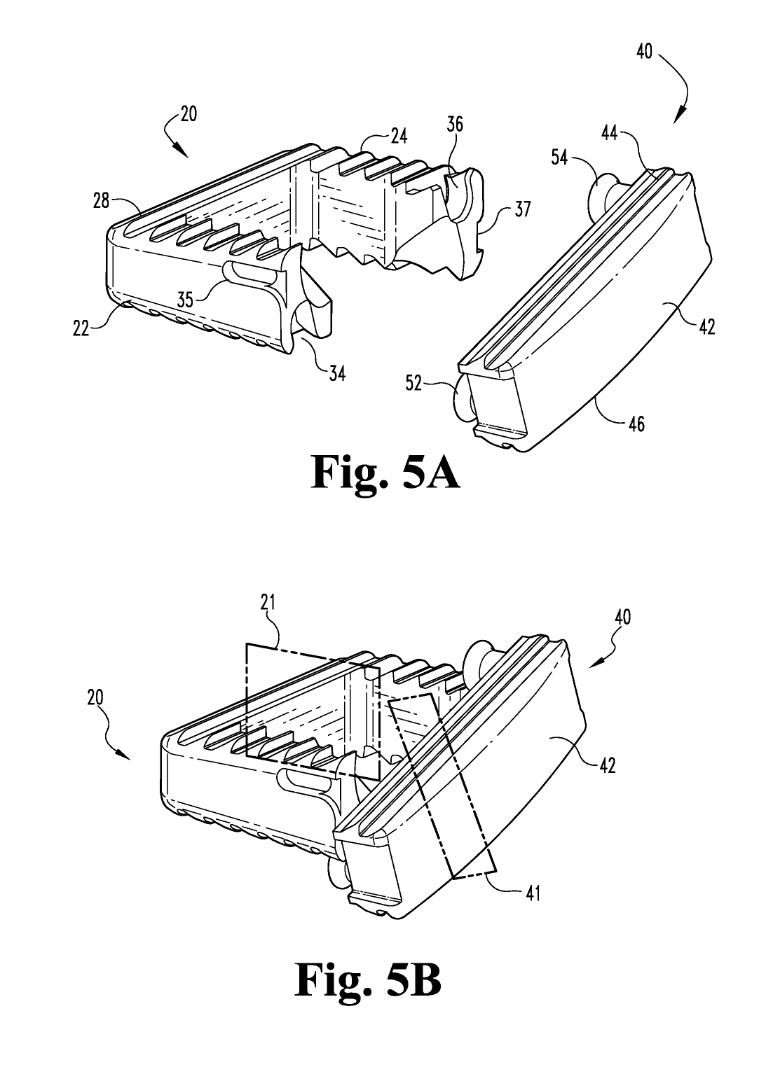

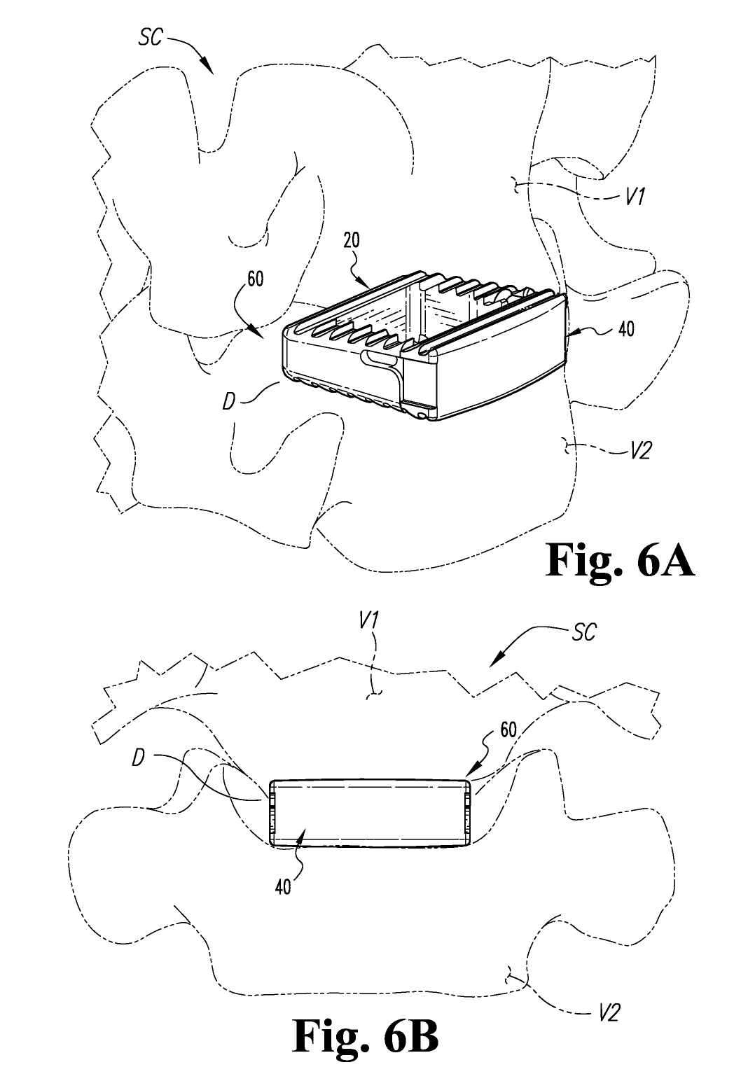

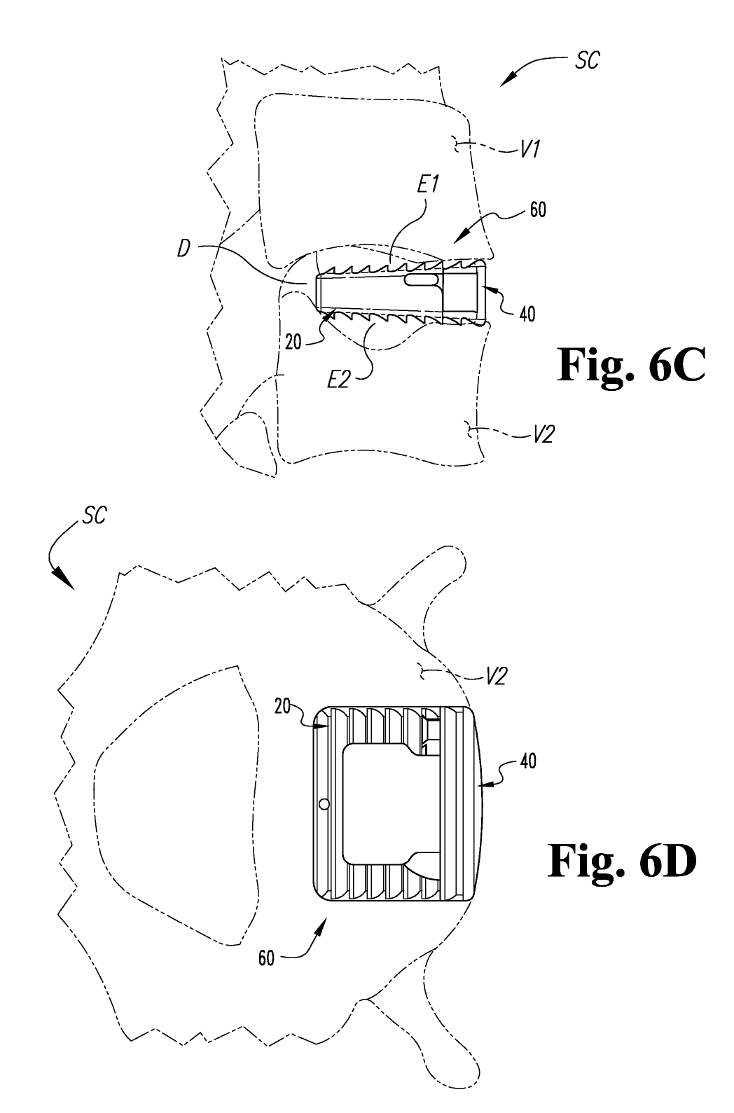

FIG. 1 illustrates a perspective view of an interbody implant element 20 positionable on the vertebral endplates between adjacent vertebral bodies to replace either a spinal disc space to fuse a single motion level or multiple disc spaces and associated vertebra or vertebrae in a corpectomy procedure to fuse multiple levels. FIG. 2 illustrates one embodiment of an add-on element 40 that is engageable to interbody element 20 at or adjacent its trailing end to form an implant assembly 60, such as shown in FIG. 5C and FIGS. 6A-6D. The add-on elements described herein can be placed either entirely or substantially entirely in the disc space, and/or the add-on element can include a portion that extends extradiscally along one or of the vertebrae outside the disc space. The extradiscally extending portion of the add-on elements can be recessed in the anterior faces of the adjacent vertebrae to provide a low or no profile configuration, or positioned on the outer facing surfaces of the adjacent vertebrae. The add-on elements can include features to engage one or more of the endplates of the adjacent vertebrae, and/or receive bone anchors that extend through the add-on element to secure the implant assembly to the vertebrae.

Interbody element 20 comprises a cage-like body with lateral walls 22, 24 extending on opposite sides of a cavity 26 and a leading end wall 28 extending between and connecting lateral walls 22, 24. Interbody element 20 is sized to fit within the disc space between vertebral bodies V1, V2. Lateral walls 22, 24 extend from a leading end wall 28 to opposite trailing ends 23, 25. Leading end wall 28 can include a convexly rounded nose to facilitate insertion into the disc space and distraction of the vertebral bodies. Leading end wall 28 can be linear between lateral walls 22, 24 as shown, or rounded to form any suitable shape. Interbody element 20 may also include superior and inferior bone engaging surfaces 30, 32 with projections in the form of teeth, ridges or other engagement structure to enhance engagement with the adjacent vertebral endplate. In the illustrated embodiment, projections 31, 33 extend across lateral walls 22, 24 and leading end wall 28, and are spaced from one another in the direction from leading end wall 28 to trailing ends 23, 25. The bone engaging surfaces 30, 32 can be planar, convexly curved, tapered, or otherwise configured to be received between and contact at least a portion of the endplates along at least a portion of the length of interbody element 20. Interbody element 20 may also include one or more cavities or holes through its superior and inferior bone engaging surfaces and/or its lateral surfaces to facilitate bone growth through interbody element 20. Lateral walls 22, 24 each extend from leading end wall 28 to a respective one of trailing ends 23, 25, and also extending from the superior bone engaging surface 30 to the inferior bone engaging surface 32. The lateral walls 22, 24 can be parallel to one another, or tapered relative to one another to converge or diverge toward the leading end wall 28. The laterally facing surfaces of the walls 22, 24, 28 can be planar, concave or convex when viewed from above or below; planar, concave or convex from the superior bone engaging surface 30 to the inferior bone engaging surface 32; or combinations thereof.

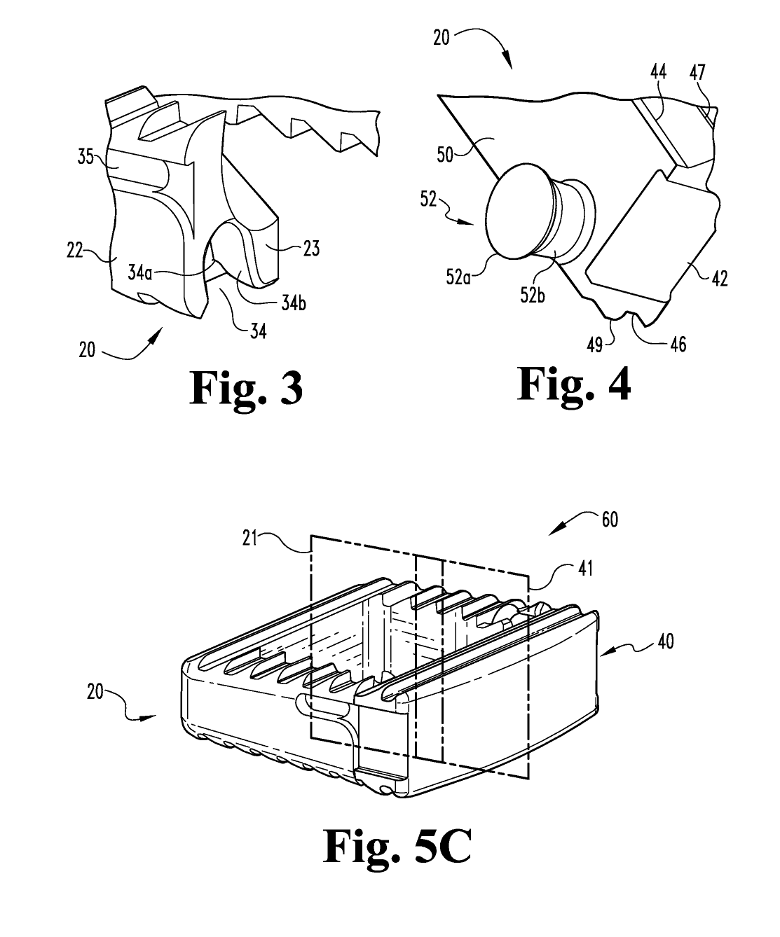

One embodiment of add-on element 40 is shown in FIG. 2. Add-on element 40 includes an elongated body 42 that is sized to extend between and connect lateral walls 22, 24 of interbody element 20. Add-on element 40 includes a superior bone engaging surface 44 and an opposite inferior bone engaging surface 46 that face and contact the endplates of the vertebrae. Bone engaging surfaces 44, 46 each include at least one projection, ridge or tooth extending therefrom. Add-on element 40 also includes an outer facing surface 48 and an opposite inner facing surface 50. In the illustrated embodiment, the bone engaging surfaces 44, 46 include a series of projections 47, 49, respectively, extending length-wise across add-on element 40 and are spaced from one another in a direction from outer surface 48 toward inner surface 50. As shown further in FIG. 4, mating elements 52, 54 project outwardly from inner surface 50 and, as shown in FIG. 3, are configured to be received in receiving portions 34, 36 formed in trailing ends 23, 25 of interbody element 20 to form an interbody spinal implant assembly 60.

Referring now further to FIGS. 5A-5C, receiving portions 34, 36 each include a side-opening, with the side opening of receiving portion 34 extending through inferior bone engaging surface 32 and the side-opening of receiving portion 36 extending through superior bone engaging surface 30. In order to secure add-on element 40 to interbody element 20, add-on element 40 is rotated in the orientation shown in FIG. 5A so that respective ones of the mating elements 52, 54 are oriented toward respective ones of the superior and inferior bone engaging surfaces 30, 32. Add-on element 40 is advanced toward interbody element 20 as shown in FIG. 5B. In this orientation, center plane 41 of add-on element 40 is oblique to center plane 21 of add-on element 20. Add-on element 40 is then rotated to move mating elements 52, 54 through the side openings of and into the respective receiving portion 34, 36, as shown in FIG. 5C. In this orientation, center plane 41 of add-on element 40 is aligned with center plane 21 of interbody element 20. In another embodiment, it is contemplated that interbody element 20 could be provided with mating elements extending therefrom and add-on element 40 includes receiving portions to receive the mating elements.

In the illustrated embodiment, mating elements 52, 54 include enlarged outer ends, as shown by enlarged outer end 52a in FIG. 4, and a recessed portion between the respective enlarged outer end and inner surface 50 of add-on element 40, as shown by recessed portion 52b of FIG. 4. Receiving portions 34, 36 each include a corresponding shape with an inner groove sufficiently wide to receive respective ones of the enlarged outer ends of the mating elements 52, 54, as shown by inner groove 34a in FIG. 3. Receiving portions 34, 36 also each include a reduced width portion sized to receive the respective recessed portions of mating elements 52, 54, as shown by reduced width portion 34b in FIG. 3. The reduced width portion 34b is narrower than the enlarged outer end 52a so that add-on element 40 cannot be pulled off of interbody element 20 unless add-on element 40 is rotated sufficiently to pass the enlarged outer ends through the corresponding side openings of receiving portions 34, 36. The enlarged outer ends 52a, 54a can maintain add-on element in abutting engagement with trailing ends 23, 25 of lateral walls 22, 24, although a spaced relation inner surface 50 and trailing ends 23, 25 is not precluded. The assembled construct provides an interbody spinal implant assembly 60 where cavity 26 is enclosed by add-on element 40 along with lateral walls 22, 24 and leading end wall 28. Bone material, bone growth material, or other suitable biocompatible material can be placed in cavity 26 to facilitate fusion.

Referring to FIGS. 6A-6C, spinal column segment SC with a vertebral body V1 along with a second vertebral body V2 and disc space D is further shown. Implant assembly 60 is positioned in disc space D. Implant assembly 60 is positioned in disc space D so its leading end wall 28 is positioned at the posterior side of disc space D and lateral walls 22, 24 extend anteriorly from leading end wall 28 to its trailing ends 23, 25 where add-on element 40 is secured. Implant assembly 60 is positioned in disc space D between vertebral bodies V1 and V2 so that when it is in its implanted orientation it contacts endplates E1 and E2. Add-on element 40 is positioned so that it lies entirely in disc space D and is aligned with or slightly posteriorly offset from the exterior anterior facing surfaces of vertebral bodies V1, V2. Thus, add-on element 40 provides a low or zero-profile face for implant assembly 60 that does not extend outwardly from disc space D or project anteriorly from the anterior facing surface of vertebrae V1, V2. This allows projections 47, 49 along bone engaging surfaces 44, 46 to engage the respective endplate E1, E2. According to one procedure, implant assembly 60 is positioned into disc space D from a direct anterior approach. As used herein, a “direct anterior approach” is an approach that is parallel or substantially parallel to the sagittal plane and thus orthogonal to or substantially orthogonal to the coronal plane. Other embodiments contemplate that implant assembly 60 is positioned into disc space D from another approach, such as a lateral, posterior, or oblique approach.

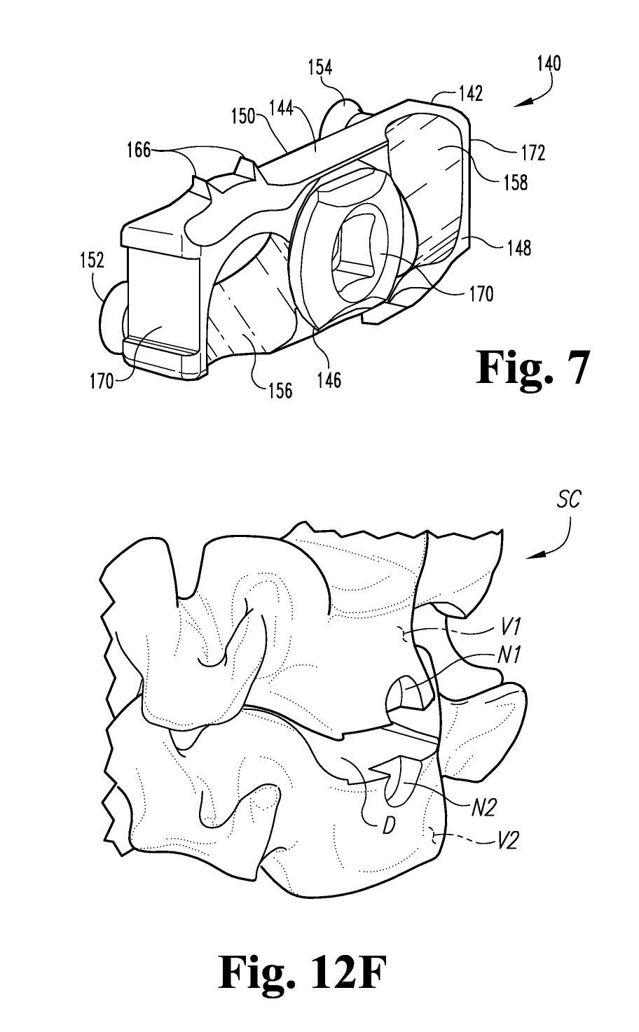

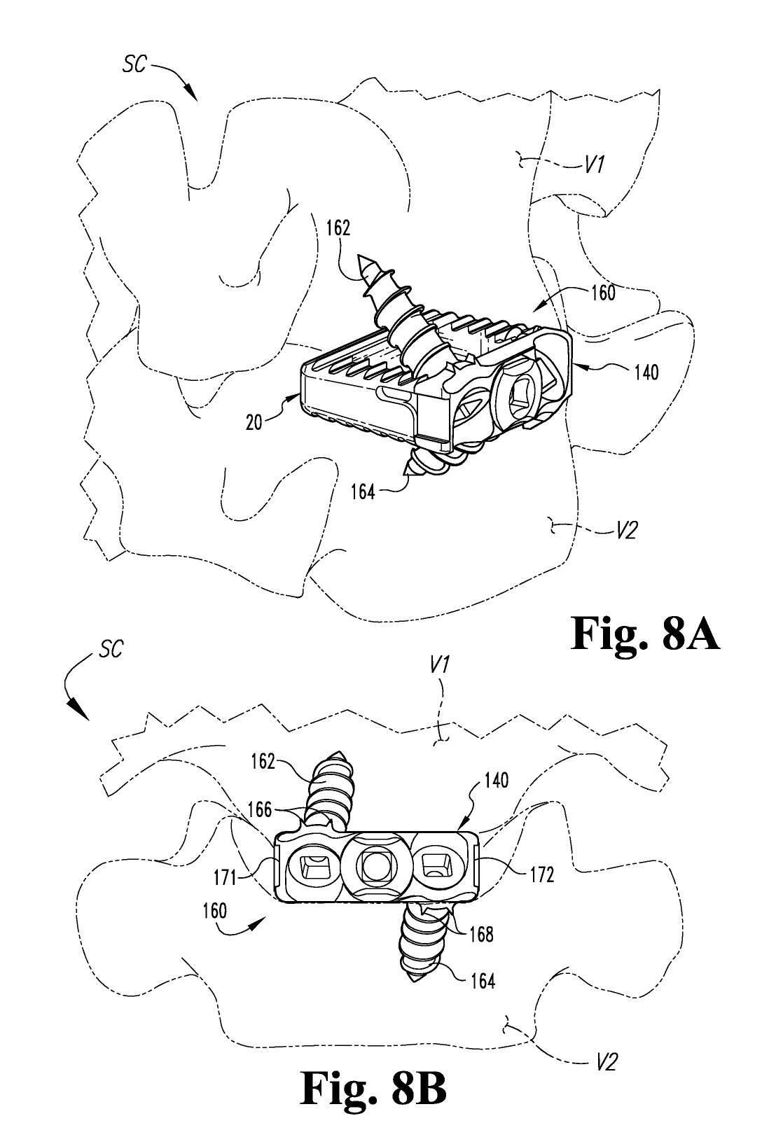

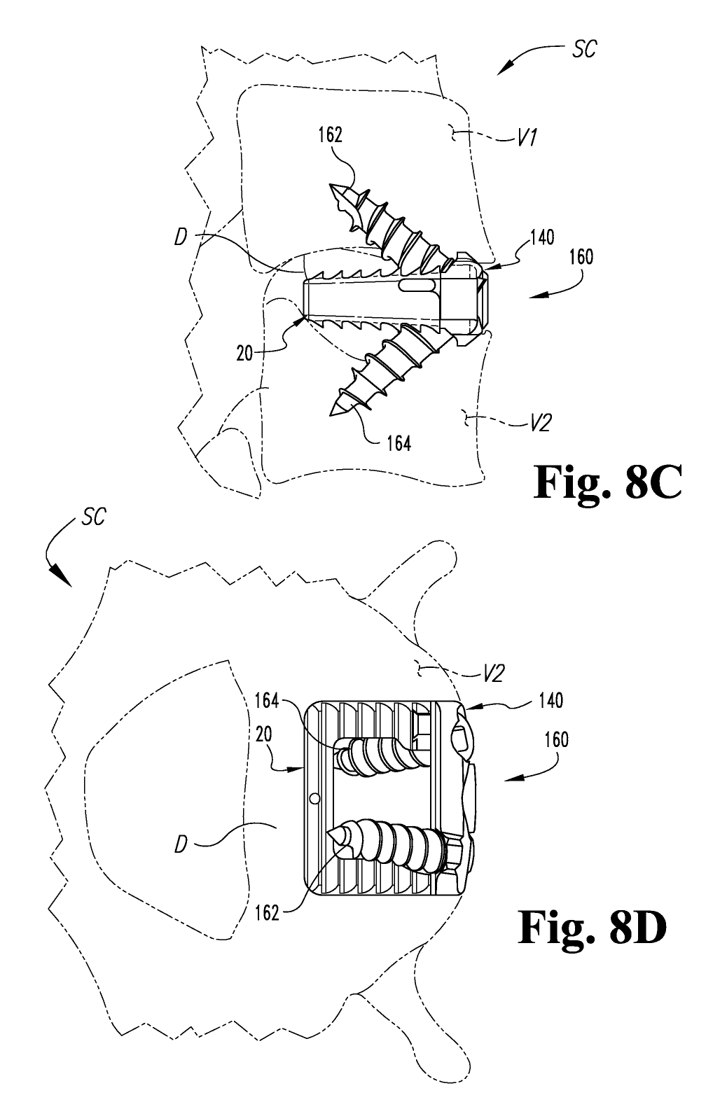

FIG. 7 shows another embodiment add-on element 140 removably attachable to interbody element 20, as shown in FIGS. 8A-8D, to form implant assembly 160. Add-on element 140 includes an elongated body 142 that is sized to extend between and connect lateral walls 22, 24 of interbody element 20. Add-on element 140 includes a superior bone engaging surface 144 and an opposite inferior bone engaging surface 146 that face and contact the endplates of respective ones of the vertebrae V1, V2. Bearing surfaces 144, 146 each include at least one projection extending therefrom. Add-on element 140 also includes an outer facing surface 148 and an opposite inner facing surface 150. Mating elements 152, 154 extend outwardly from inner surface 150 and, as shown in FIGS. 8A-8D, are configured to be received in receiving portions 34, 36 formed in trailing ends 23, 25 of interbody element 20. In addition, add-on element 140 includes first and second apertures 156, 158 located on opposite sides of the center plane of add-on element 140 and implant assembly 160 in order to receive bone anchors 162, 164, respectively, to anchor implant assembly 160 to respective ones of the vertebrae V1, V2. First aperture 156 extends from outer facing surface 148 and through inner facing surface 150, and is angled cephaladly and toward the sagittal plane so that bone anchor 162 extends into superior vertebra V1. Second aperture 158 extends from outer facing surface 148 and through inner facing surface 150, and is angled caudally and toward the sagittal plane so that bone anchor 164 extends into inferior vertebra V2. Add-on element 140 includes projections in the form of a pair of spikes 166 extending from bone engaging surface 144 aligned with first aperture 156 that extend from superior bearing surface 144 that embed into the endplate of vertebra V1. As bone anchor 162 engages vertebra V1, endplate E1 of vertebra V1 is drawn into contact with and embedded onto spikes 166. Add-on element 140 also includes projections in the form of a pair of spikes 168 extending from inferior bearing surface 146 that are aligned with second aperture 156 that embed into vertebra V2. As bone anchor 164 engages vertebra V2, endplate E2 of vertebra V2 is drawn into contact with and embedded onto spikes 168. A retaining element 170 is secured to add-on element 140 and positioned between apertures 156, 158 on outer facing surface 148. Retaining element 170 can be rotated relative to add-on element 140 between a first a position to allow bone anchor insertion into apertures 156, 158 and a second position where retaining element 170 overlaps the inserted bone anchors to prevent or resist anchor backout from apertures 156, 158.

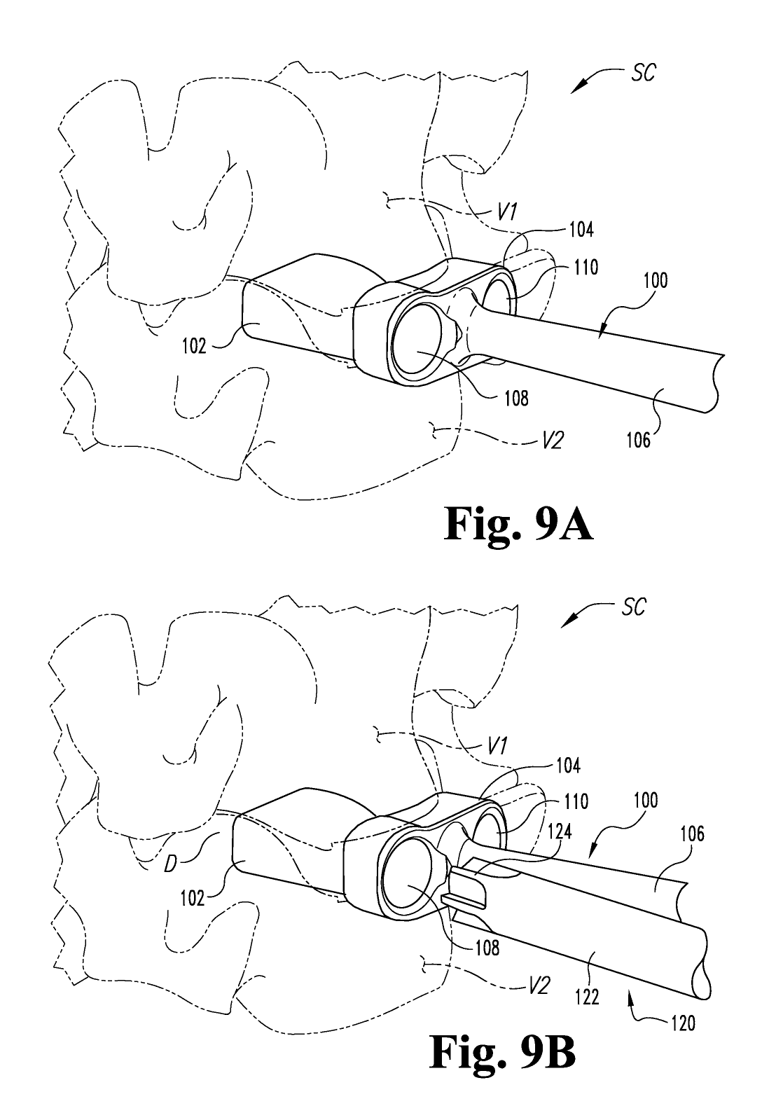

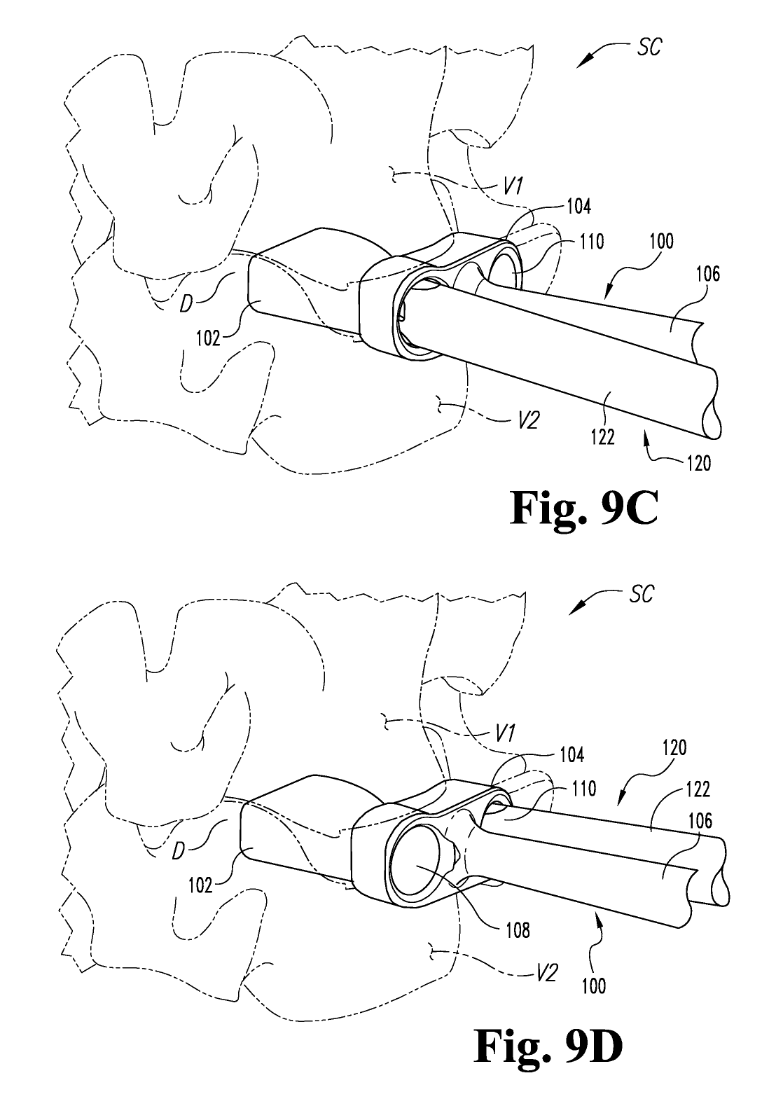

FIGS. 9A-9D show spinal column segment SC and a reaming guide 100 and reamer 120 to prepare vertebrae V1, V2 to receive anchors extending from add-on element 140. Reaming guide 100 includes a body 102 positionable in disc space D, a guide flange 104 at the proximal end of body 102, and a central shaft 106 extending proximally from guide flange 104 to a proximal handle (not shown). Guide flange 104 includes a first angle passage 108 at one side of shaft 106 and a second angled passage 110 at the opposite side of shaft 106. Reamer 120 includes an elongated shaft 122 extending from a proximal handle (not shown) to a distal reaming head 124. Reaming head 124 is positionable into respective ones of the angled passages 108, 110 and guided into the corresponding vertebra V1, V2 to form a path for insertion of and to receive add-on element 140 in disc space D so that add-on element does not project anterior from disc space D, forming a low or no-profile arrangement when implant assembly 160 is positioned in disc space D.

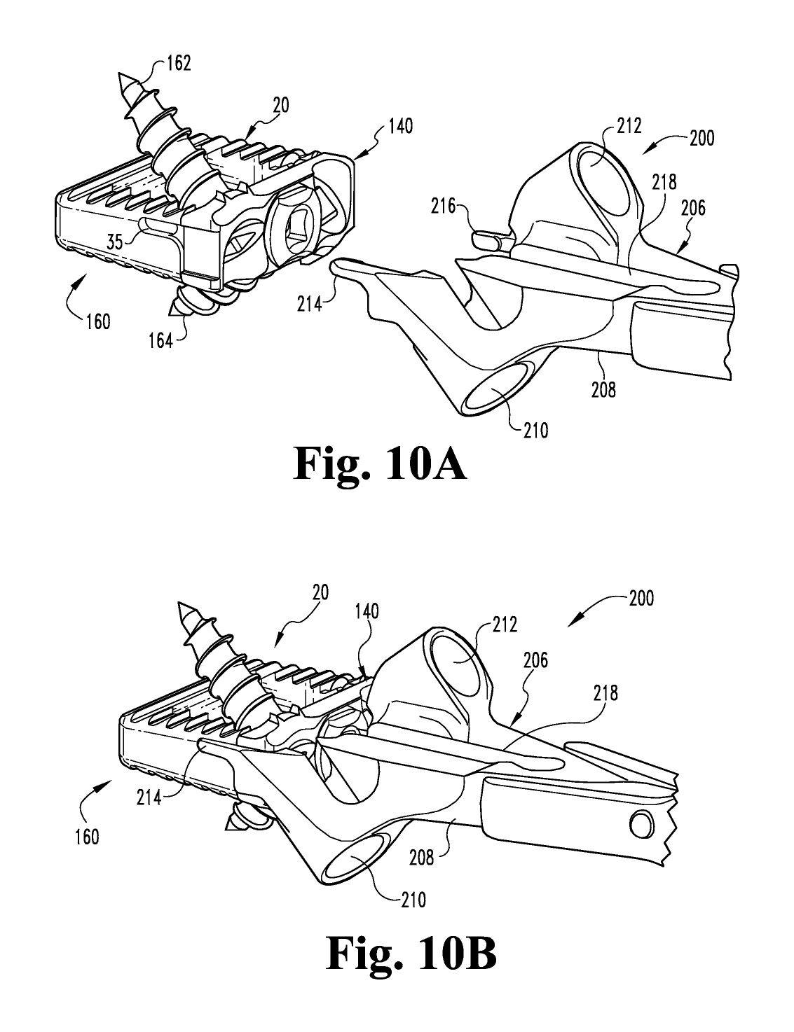

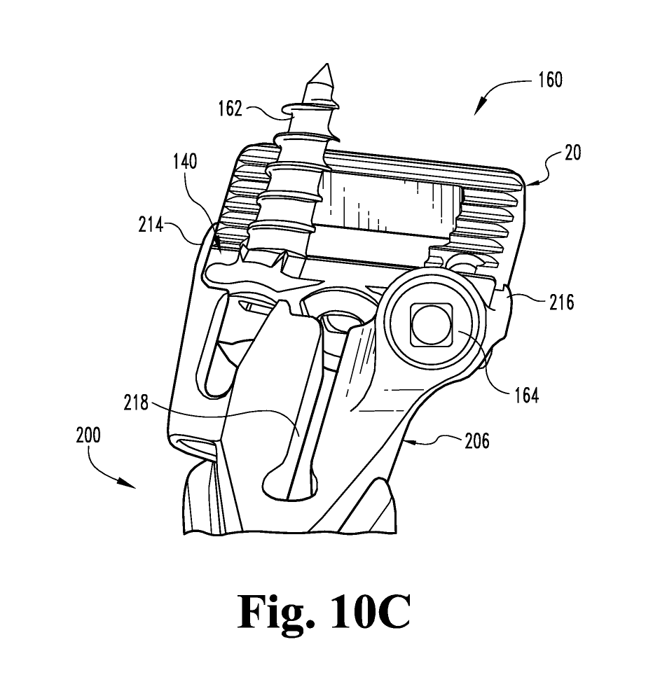

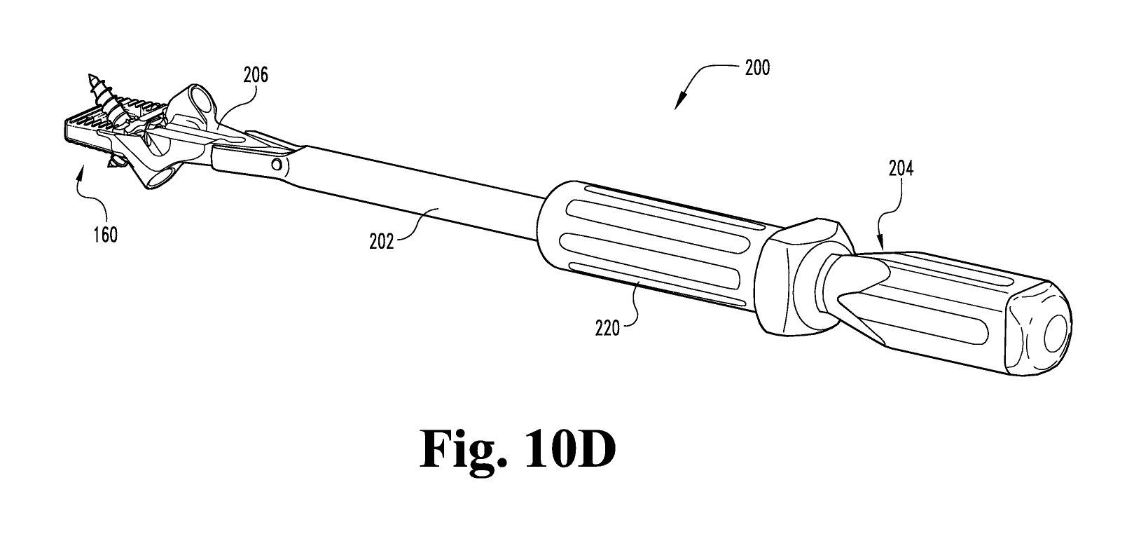

FIGS. 10A-10D show an inserter instrument 200 engaged to implant assembly 160 including add-on element 140 and interbody element 20. Inserter instrument 200 includes an elongated shaft 202 extending between a proximal handle 204 and an insertion head 206 at the distal end of shaft 202. Insertion head 206 can be removably coupled to shaft 202 to allow assembly of another embodiment insertion head configured for other embodiment add-on elements. However, permanent attachment of insertion head 206 to shaft 202 is not precluded. Insertion head 206 includes a guide body 208 extending longitudinally in alignment with shaft 202. Body 202 includes a first angled passage 210 and a second angled passage 212 on opposite sides thereof that align with and form proximal extensions of respective ones of the bone anchor apertures 156, 158 of add-on element 140. Insertion head 206 also includes opposite fingers 214, 216 that are positioned in grooves 171, 172 (see also FIGS. 7 and 8B) on opposite sides of add-on element 140 and into grooves 35, 37 (see also FIG. 5A) along the laterally outer facing surfaces of lateral walls 22, 24 of interbody element 20. Positioning of fingers 214, 216 in the grooves ensures that passages 210, 212 are properly positioned and aligned relative to the apertures 156, 158 of add-on element 140. Inserter instrument 200 also includes a longitudinally movable member 220 that is movable along shaft 202 to compress guide body 208 along longitudinal slot 218 and move fingers 214, 216 toward one another to grip implant assembly 160 therebetween. With the interbody element 20 and add-on element 140 positioned in the space between the vertebrae, angled passages 210, 212 are used to guide bone anchor preparation instruments and/or bone anchors 162, 164 through apertures 156, 158 at the desired angle into the respective vertebra V1, V2.

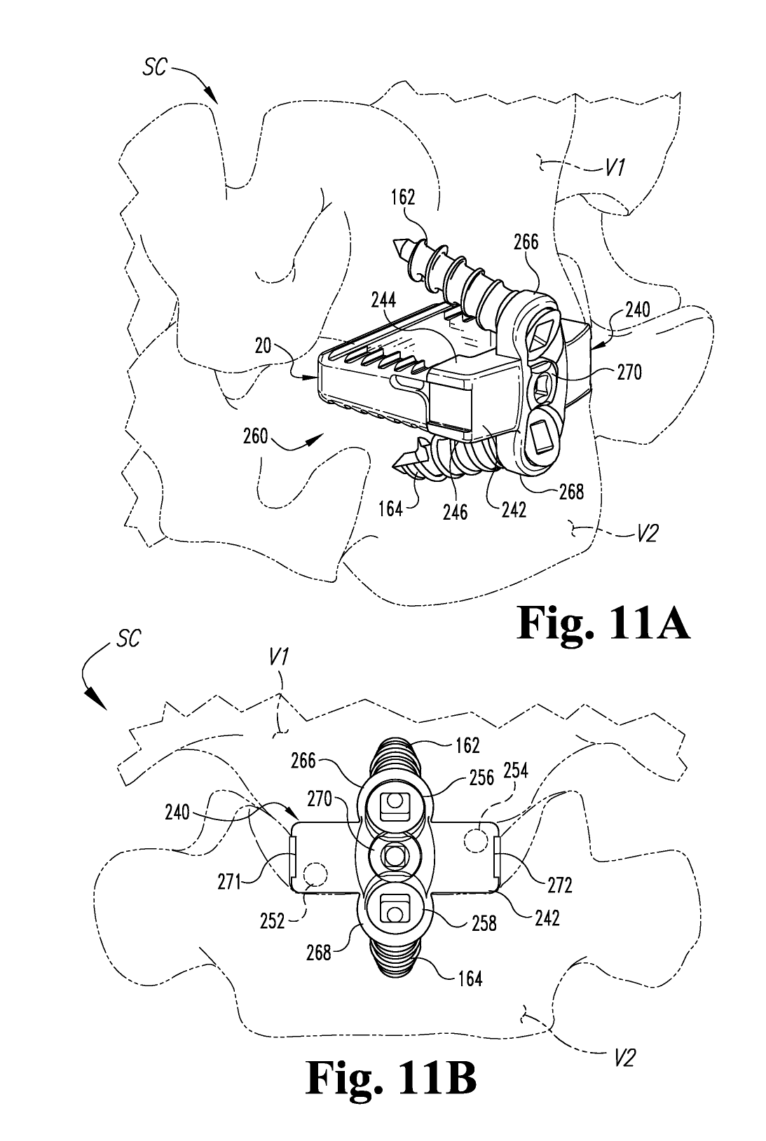

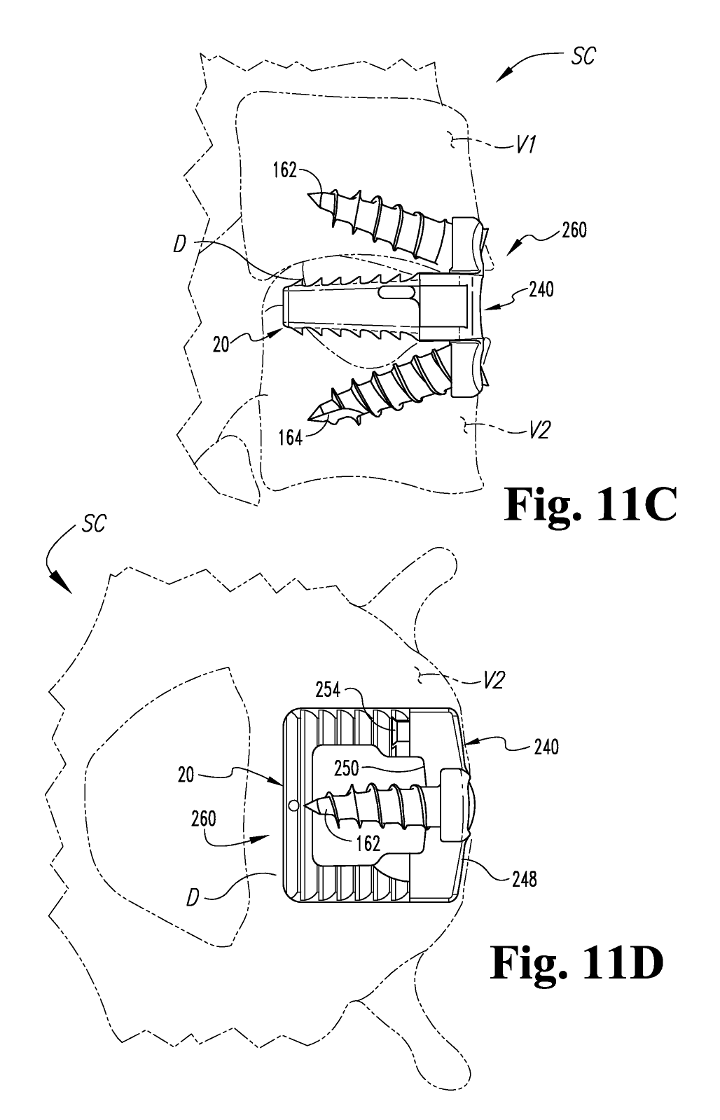

FIGS. 11A-11D show another embodiment add-on element 240 removably attachable to interbody element 20 to form implant assembly 260. Add-on element 240 includes an elongated body 242 that is sized to extend between and connect lateral walls 22, 24 of interbody element 20. Add-on element 240 includes a superior bone engaging surface 244 and an opposite inferior bone engaging surface 246 that face and contact the endplates of respective ones of the vertebrae V1, V2. Engaging surfaces 244, 246 each include at least one plate-like projection 266, 268 extending therefrom. Add-on element 240 also includes an outer facing surface 248 and an opposite inner facing surface 250. Mating elements 252, 254 extend outwardly from inner surface 250 and are configured to be received in receiving portions 34, 36 formed in trailing ends 23, 25 of interbody element 20. In addition, add-on element 240 includes first and second apertures 256, 258 located through respective ones of the projections 266, 268 on the center plane of add-on element 240 and implant assembly 260 in order to receive bone anchors 162, 164, respectively, to anchor implant assembly 260 to respective ones of the vertebrae V1, V2. First aperture 256 is angled cephaladly and on, parallel to, or generally parallel to the sagittal plane so that bone anchor 162 extends into superior vertebra V1. Second aperture 258 is angled caudally and on, parallel to, or generally parallel to the sagittal plane so that bone anchor 164 extends into inferior vertebra V2. A retaining element 270 is secured to add-on element 240 and positioned between apertures 256, 258 on outer facing surface 248. Retaining element 270 can be rotated relative to add-on element 240 between a first a position to allow bone anchor insertion into apertures 256, 258 and a second position where retaining element 270 overlaps the inserted bone anchors to prevent or resist anchor backout from apertures 256, 258.

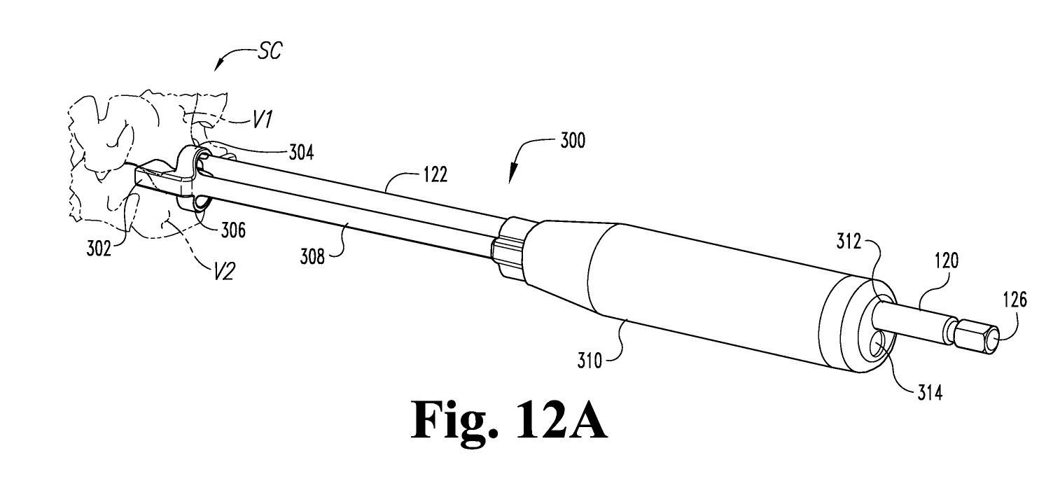

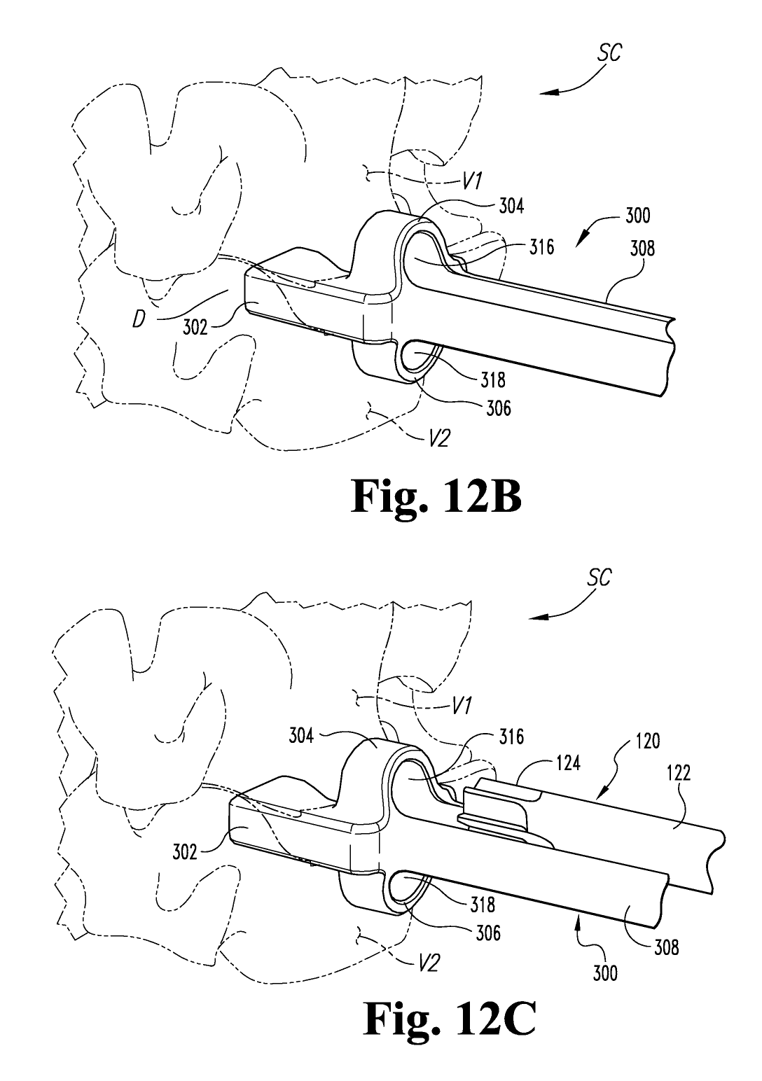

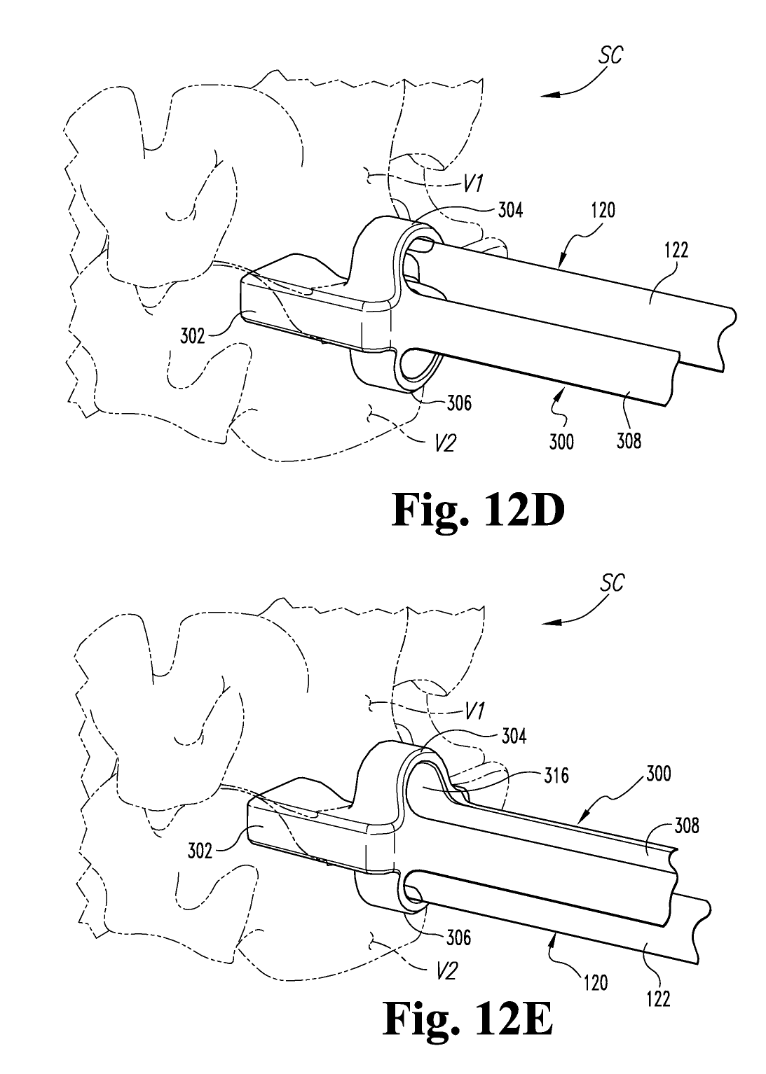

FIGS. 12A-12F show spinal column segment SC and another embodiment reaming guide 300 with reamer 120 to prepare vertebrae V1, V2 to receive projections 266, 268 of add-on element 240 so that projections 266, 268 are recessed into the anterior faces of respective ones of vertebrae V1, V2 to form a low or no-profile implant assembly 260 that does not projection from the anterior faces of vertebrae V1, V2. Reaming guide 300 includes a body 302 positionable in disc space D, a pair of guide flanges 304, 306 extending superiorly and inferiorly, respectively, from body 302, and a central shaft 308 extending proximally from body 302 to a proximal handle 310. Handle 310 includes a superior passage 312 and an inferior passage 314 to receive shaft 122 of reamer 120 therethrough. The proximal end 126 of reamer 120 includes a boss to receive a T-handle or other device to facilitate rotation of reamer 120. Guide flange 304 includes a first passage 316 extending therethrough and guide flange 306 includes a second passage 318 extending therethrough. Reamer 120 includes elongated shaft 122 extending to distal reaming head 124. Reaming head 124 is positionable into respective ones of the passages 316, 318 and guided into the corresponding vertebra V1, V2 to form a notch N1, N2 as shown in FIG. 12F for receiving the projections 266, 268 of add-on element 240 in recessed relation to the anterior faces of vertebrae V1, V2, as shown in FIGS. 11A-11D.

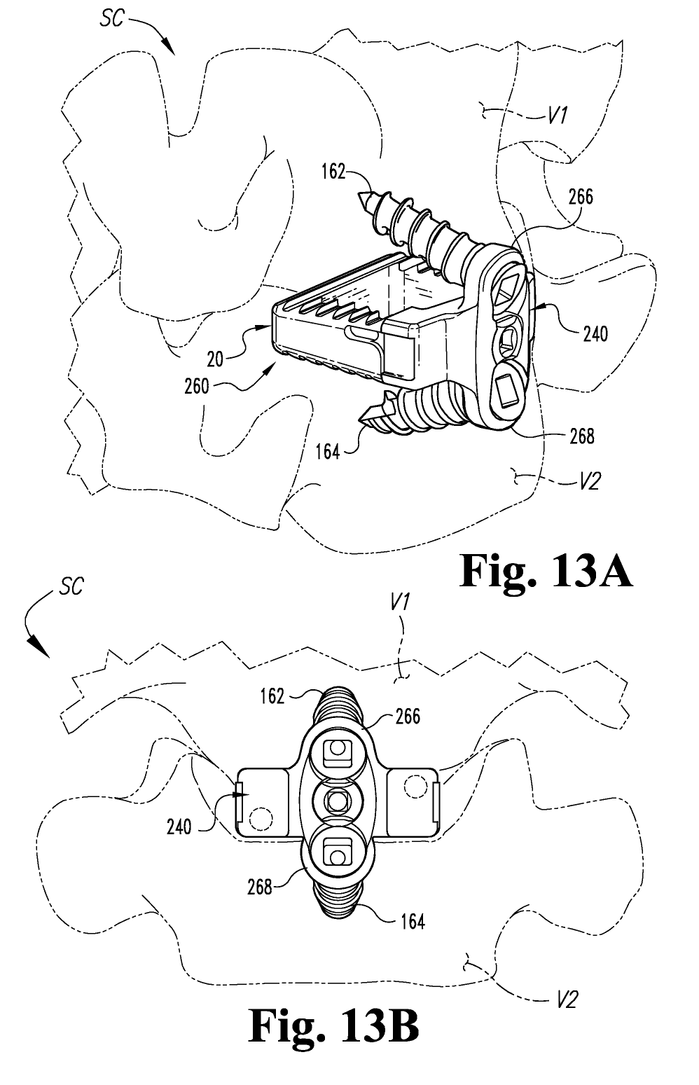

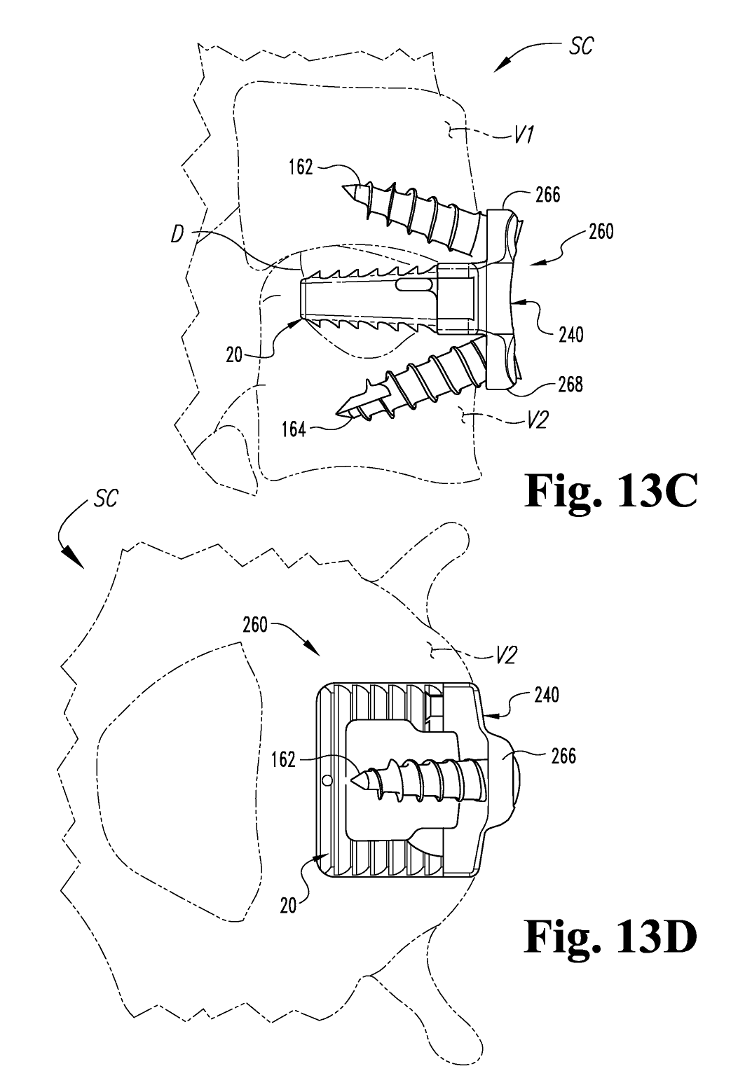

FIGS. 13A-13D show add-on element 240 removably attached to interbody element 20 to form implant assembly 260 positioned in a positive profile position relative to vertebrae V1, V2. In the positive profile position, projections 266, 268 project anteriorly from the anterior faces of vertebrae V1, V2, while bone engaging surfaces 244, 246 directly engage the endplates of respective ones of vertebrae V1, V2.

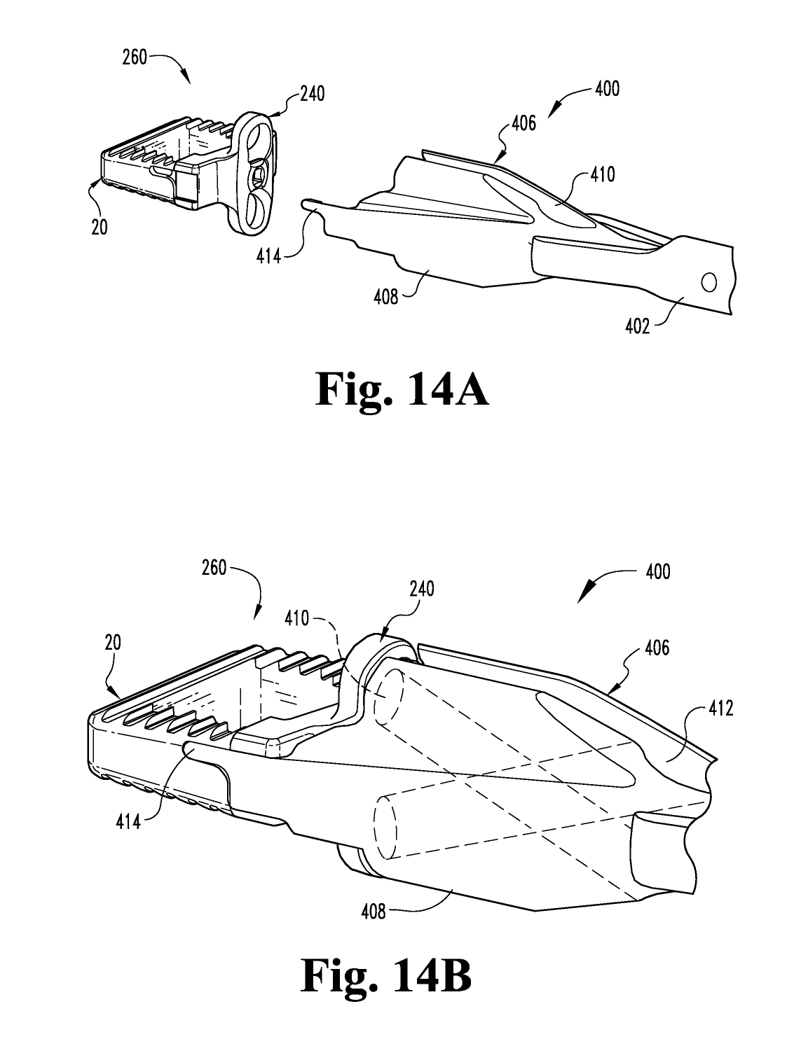

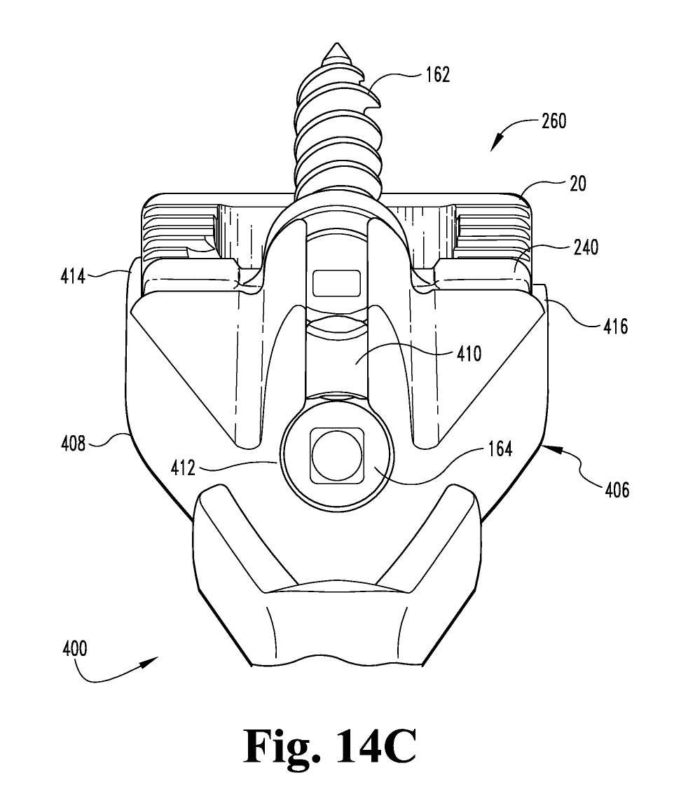

FIGS. 14A-14C show an inserter instrument 400 engaged to implant assembly 260 including add-on element 240 and interbody element 20. Inserter instrument 400 includes an elongated shaft 402 extending between a proximal handle like that shown with respect to inserter instrument 200 discussed above. Inserter instrument 400 includes an insertion head 406 at the distal end of shaft 402. Insertion head 406 can be removably coupled to shaft 402 to allow assembly of another embodiment insertion head configured for other embodiment add-on elements. However, permanent attachment of insertion head 406 to shaft 402 is not precluded. Insertion head 406 includes a body 408 extending longitudinally in alignment with shaft 402. Body 408 includes a first angled passage 410 and a second angled passage 412 the cross-over one another in body 408 so that when assembled with implant assembly 260, passage 410 opens distally at aperture 256 and proximally on an inferior side of body 408 and so that passage 412 opens distally at aperture 258 and proximally on a superior side of body 408. Passages 410, 412 align with and form proximal extensions of respective ones of the bone anchor apertures 256, 258 of add-on element 240 to guide insertion of bone anchor preparation instruments and/or bone anchors 162, 164. Insertion head 406 also includes opposite fingers 414, 416 that are positioned in grooves 271, 272 on opposite sides of add-on element 240 and into grooves 35, 37 along the laterally outwardly facing surfaces of lateral walls 22, 24 of interbody element 20. Positioning of fingers 414, 416 in the grooves ensures that passages 410, 412 are properly positioned and aligned relative to the apertures 256, 258 of add-on element 240. Inserter 400 also includes a longitudinally movable member (like member 220 of inserter 200) that is movable along shaft 402 to compress body 408 along slot 418 and move fingers 414, 416 toward one another to grip implant assembly 260 therebetween. With the interbody element 20 and add-on element 240 positioned in the space between the vertebrae, angled passages 410, 412 are used to guide bone anchors 162, 164 through apertures 256, 258 at the desired angle into the respective vertebra V1, V2.

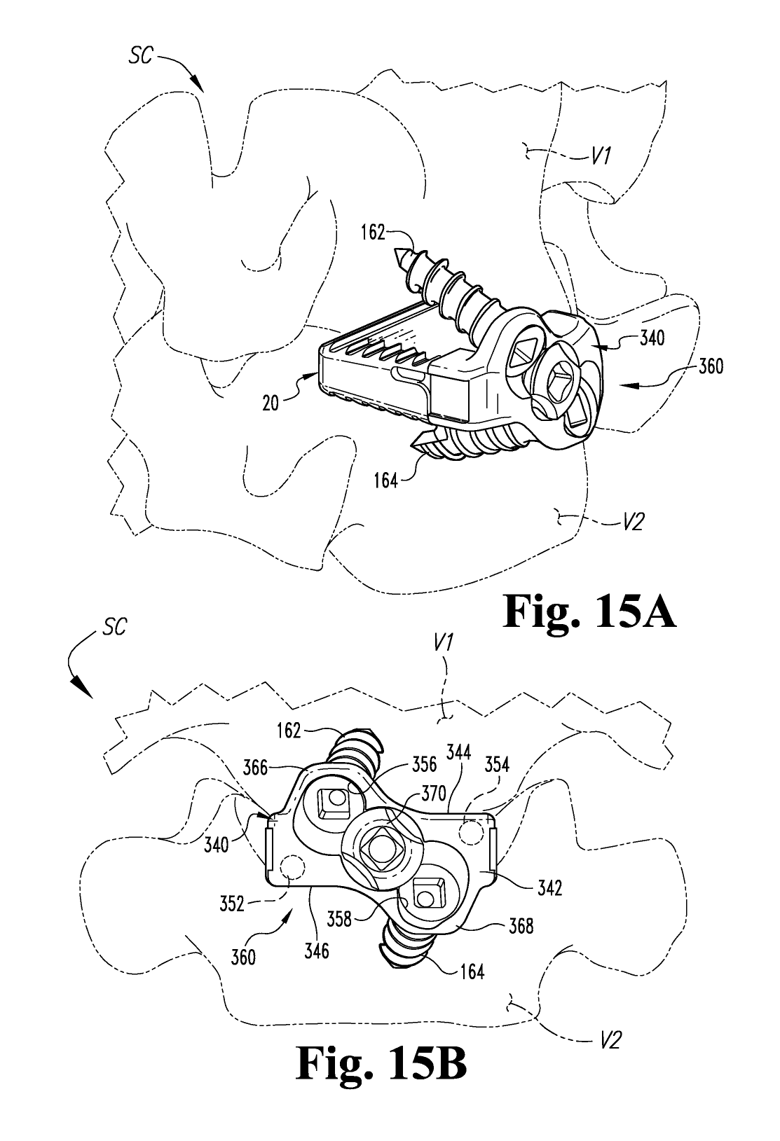

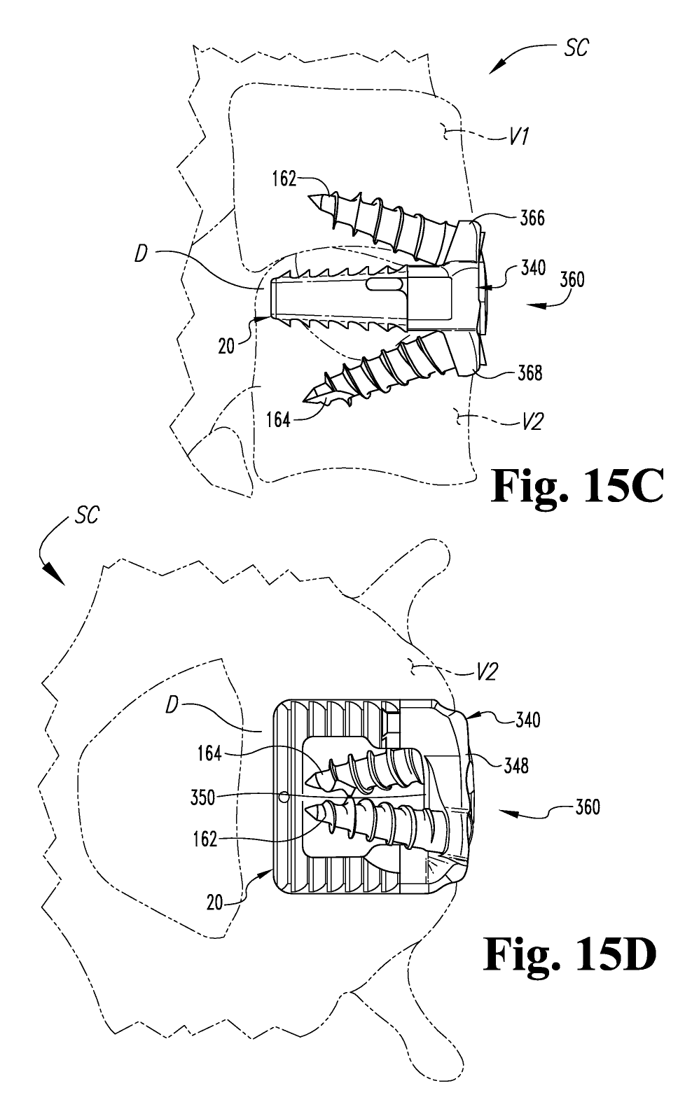

FIGS. 15A-15D show another embodiment add-on element 340 removably attachable to interbody element 20 to form implant assembly 360. Add-on element 340 includes an elongated body 342 that is sized to extend between and connect lateral walls 22, 24 of interbody element 20. Add-on element 340 includes a superior bone engaging surface 344 and an opposite inferior bone engaging surface 346 that face and contact the endplates of respective ones of the vertebrae V1, V2. Engaging surfaces 344, 346 each include at least one plate-like projection 366, 368, respectively, extending therefrom. Add-on element 340 also includes an outer facing surface 348 and an opposite inner facing surface 350. Mating elements 352, 354 extend outwardly from inner surface 350 and are configured to be received in receiving portions 34, 36 formed in ends 23, 25 of interbody element 20. In addition, add-on element 340 includes first and second apertures 356, 358 located through respective ones of the projections 366, 368 that are offset on opposite sides of the center plane of add-on element 340 and implant assembly 360 in order to receive bone anchors 162, 164, respectively, to anchor implant assembly 360 to respective ones of the vertebrae V1, V2. First aperture 356 is angled cephaladly and toward the sagittal plane so that bone anchor 162 extends into superior vertebra V1. Second aperture 358 is angled caudally and toward the sagittal plane so that bone anchor 164 extends into inferior vertebra V2. A retaining element 370 is secured to add-on element 340 and positioned between apertures 356, 358 on outer facing surface 348. Retaining element 370 can be rotated relative to add-on element 340 between a first a position to allow bone anchor insertion into apertures 356, 358 and a second position where retaining element 370 overlaps the inserted bone anchors to prevent or resist anchor backout from apertures 356, 358.

With add-on element 340, projections 366, 368 are angled anteriorly relative to body 342, as shown most clearly in FIG. 15C, so that when bone anchors 162, 164 are inserted normal to the anterior facing surface of projections 366, 368, bone anchors 162, 164 are angled superiorly and inferiorly, respectively. This allows bone anchors 162, 164 to be inserted through apertures 356, 358 with a freehand technique. In addition, notches can be formed in the anterior faces of vertebrae V1, V2 so that projections 366, 368 are recessed into the outer facing surfaces of vertebrae V1, V2 to minimize or eliminate the outward projection of add-on element 340 from the anterior facing surfaces of vertebrae V1, V2.

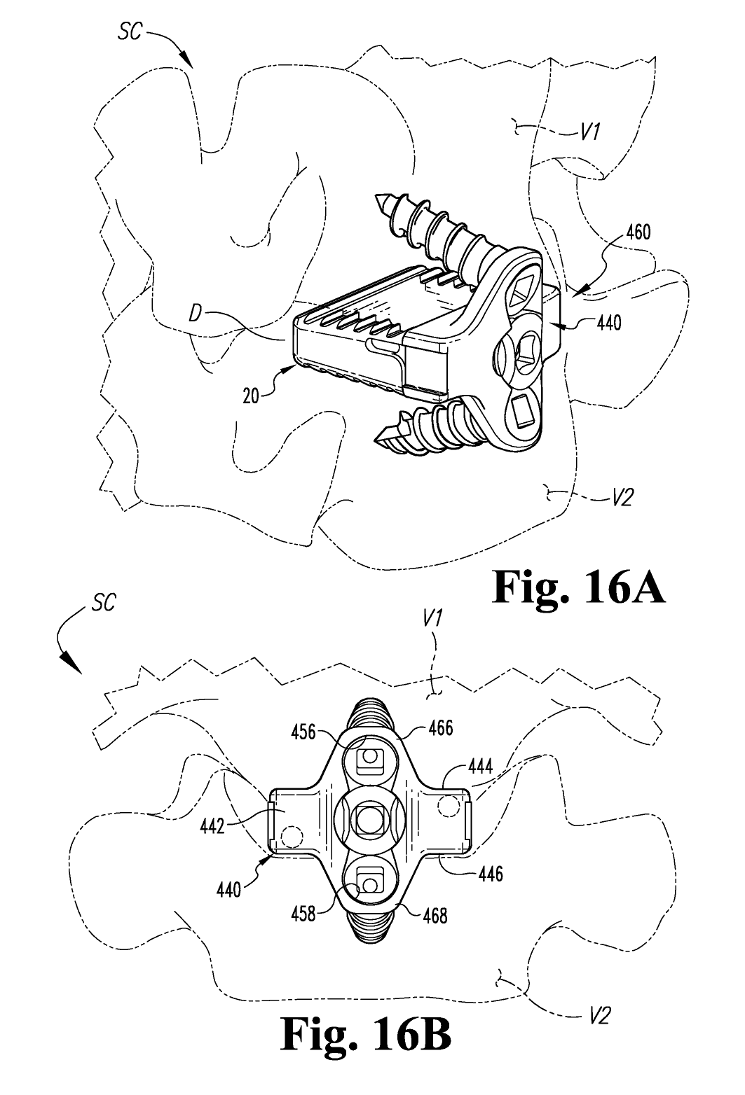

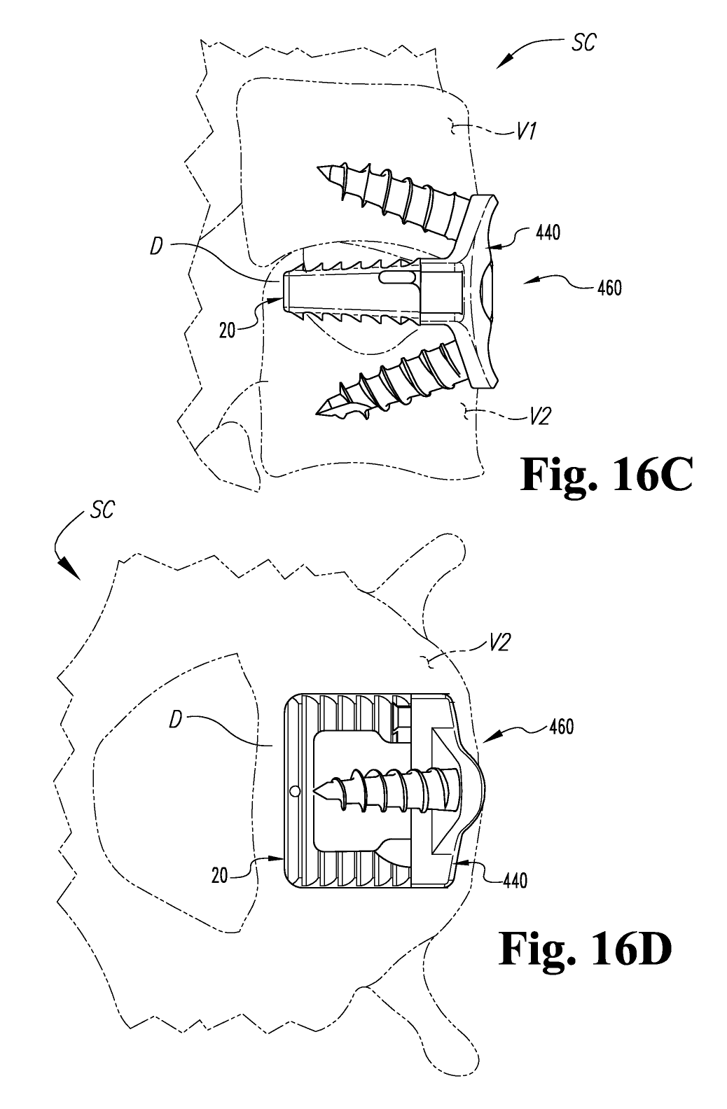

FIGS. 16A-16D show another embodiment add-on element 440 removably attachable to interbody element 20 to form implant assembly 260. Add-on element 440 is similar to add-on element 240 but includes at least one plate-like projection 466, 468 extending form respective ones of the superior and inferior bone engaging surfaces 444, 446 that are angled anteriorly relative to body 442, as shown most clearly in FIG. 16C, so that when bone anchors 162, 164 are inserted normal to the anterior facing surface of projections 466, 468, bone anchors 162, 164 are angled superiorly and inferiorly, respectively. This allows bone anchors 162, 164 to be inserted through apertures 456, 458 with a freehand technique without risk of penetrating the endplates of the vertebrae V1, V2. In addition, notches can be formed in the anterior faces of vertebrae V1, V2 so that projections 466, 468 are recessed into the outer facing surfaces of vertebrae V1, V2 to minimize or eliminate the outward projection of add-on element 440 from vertebrae V1, V2. Add-on element 440 includes first and second apertures 456, 458 located through respective ones of the projections 466, 468 that are aligned with one another on the center plane of add-on element 440 and implant assembly 460 in order to receive bone anchors 162, 164, respectively, to anchor implant assembly 460 to respective ones of the vertebrae V1, V2. First aperture 456 is angled cephaladly and on, parallel to, or generally parallel to the sagittal plane so that bone anchor 162 extends into superior vertebra V1. Second aperture 458 is angled caudally and on, parallel to, or generally parallel to the sagittal plane so that bone anchor 164 extends into inferior vertebra V2.

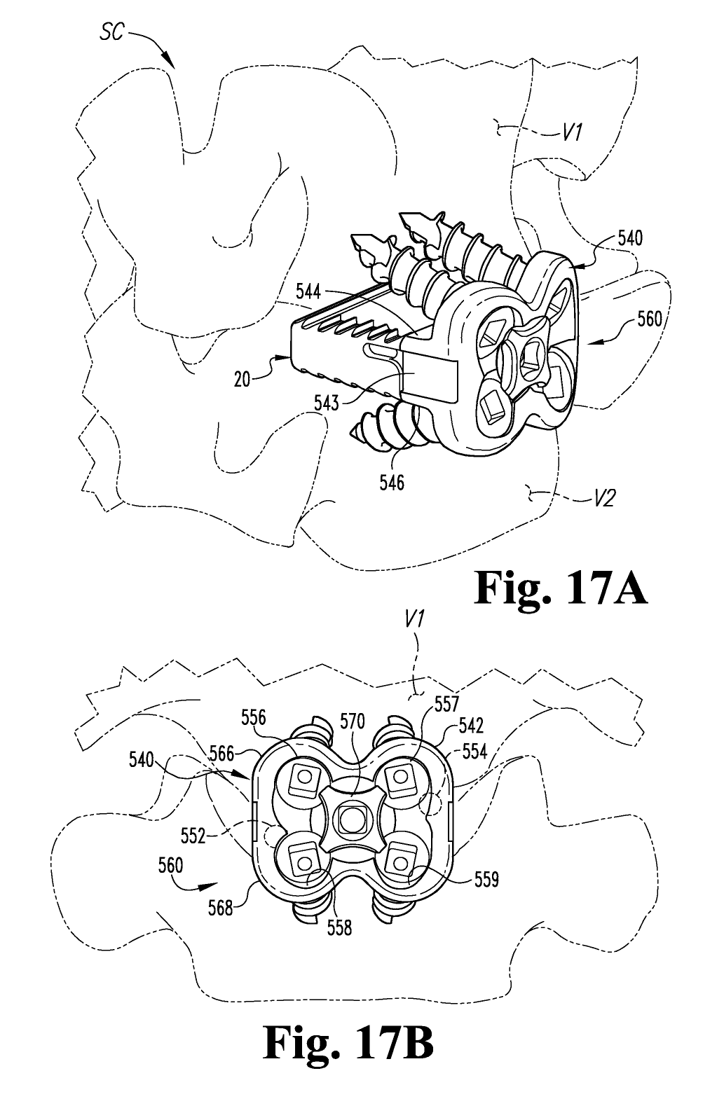

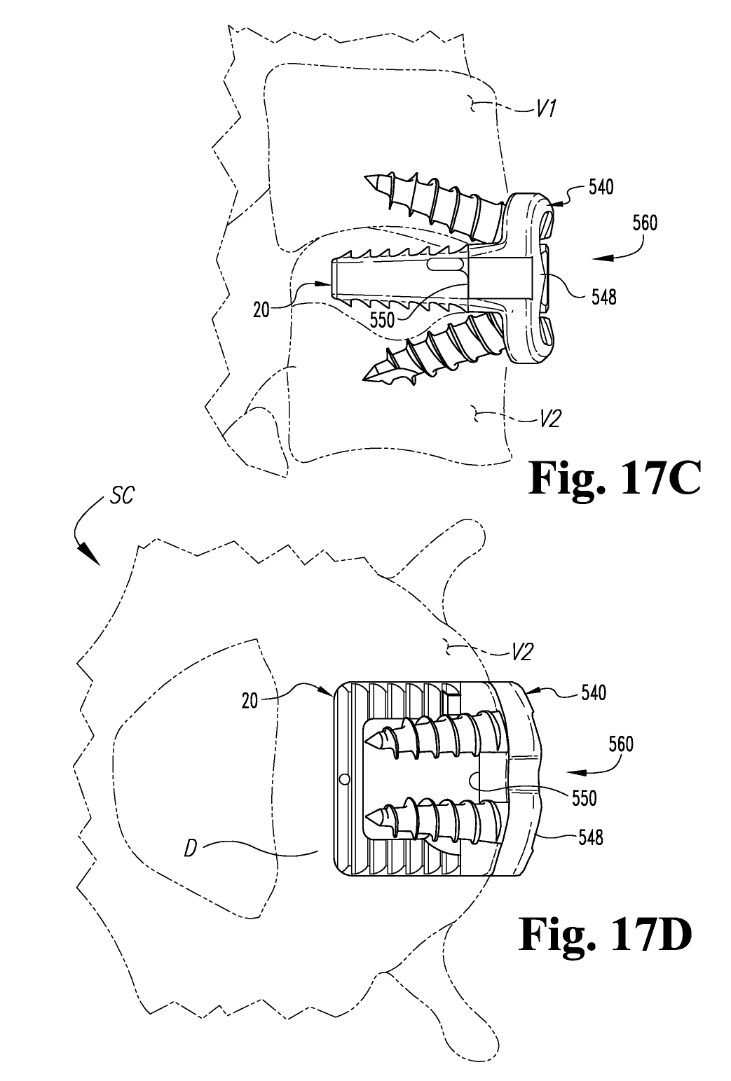

FIGS. 17A-17D show another embodiment add-on element 540 removably attachable to interbody element 20 to form implant assembly 560. Add-on element 540 includes a plate-shaped body 542 with an intradiscal portion 543 that is sized to extend between and connect lateral walls 22, 24 of interbody element 20. Intradiscal portion 543 includes a superior bone engaging surface 544 and an opposite inferior bone engaging surface 546 that face and contact the endplates of respective ones of the vertebrae V1, V2. Engaging surfaces 544, 546 each include at least one plate-like projection 566, 568 extending therefrom. Add-on element 540 also includes an outer facing surface 548 and an opposite inner facing surface 550. Mating elements 552, 554 extend outwardly from inner surface 550 and are configured to be received in receiving portions 34, 36 formed in trailing ends 23, 25 of interbody element 20. In addition, add-on element 540 includes first and second apertures 556, 557 located through projection 566, and third and fourth apertures 558, 559 located through projection 568. Apertures 556, 557 are offset from one another on opposite sides of the center plane of add-on element 540 and apertures 558, 559 are offset from one another on opposite sides of the center plane of add-on element 540. Each of the apertures receives a bone anchor to anchor implant assembly 560 to respective ones of the vertebrae V1, V2. First and second apertures 556, 557 are angled cephaladly and toward one another so that the bone anchors extend superiorly into superior vertebra V1 and toward the sagittal plane. Third and fourth apertures 558, 559 are angled caudally and toward one another so that the bone anchors extend caudally into inferior vertebra V2 and toward the sagittal plane. A retaining element 470 is secured to add-on element 440 and positioned centrally between apertures 556, 557, 558, 559 on outer facing surface 548. Retaining element 570 can be rotated relative to add-on element 540 between a first a position to allow bone anchor insertion into apertures 556, 557, 558, 559 and a second position where retaining element 570 overlaps the inserted bone anchors to prevent or resist anchor backout from apertures 556, 557, 558, 559 as shown in FIG. 17B.

FIGS. 17A-17D show add-on element 540 removably attached to interbody element 20 to form implant assembly 560 positioned in a positive profile position relative to vertebrae V1, V2. In the positive profile position, projections 566, 568 project anteriorly from the anterior faces of vertebrae V1, V2. In addition, projections 566, 568 are angled anteriorly away from outer facing surface 548 so that the bone anchors can be guided through the apertures and into vertebrae V1, V2 without penetrating the endplates of vertebrae V1, V2.

Reamer guides similar to the one shown in FIGS. 12A-12E are contemplated that are configured to be used in conjunction with the implant assembly embodiments shown in FIGS. 13A-13d, FIGS. 15A-15D, FIGS. 16A-16D, and FIGS. 17A-17D. Inserter instruments similar to the one shown in FIGS. 14A-14C are contemplated that are configured to be used in conjunction with the implant assembly embodiments shown in FIGS. 15a-15D, FIGS. 16A-16D, and FIGS. 17A-17D.

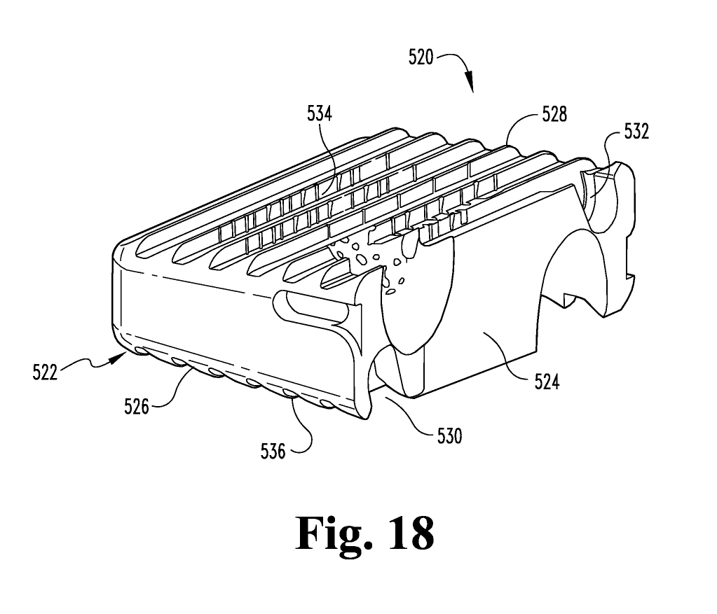

FIG. 18 illustrates another embodiment interbody element 520 that is attachable with any of the add-on elements described herein. Interbody element 520 is similar to interbody element 20 discussed above, but includes a body 522 with a trailing end surface 524 that extends between and connects lateral surfaces 526, 528. Trailing end surface 524 includes receiving portions 530, 532 therein that are identical to receiving portions 34, 36 of interbody element 20 to removably receive mating elements from the add-on element attached thereto. Body 522 lacks a cavity opening at superior and inferior bone engaging surfaces 534, 536, although inclusion of the same is not prohibited. In one specific embodiment, interbody element 520 is made from tissue such as bone, or tissue substitute that enhances bone growth and/or bonding during fusion. The add-on element to be secured to interbody element 520 can be made from a stronger material removably engaged to interbody element 520 to provide a platform for more solid engagement with the bone anchor to be secured to vertebrae V1, V2. It should be understood, however, the interbody elements and add-on elements discussed herein can be made from any suitable biocompatible material, and can be made from the same material or from different material to enhance the functionality of each element. Examples of suitable materials include titanium, titanium alloys, cobalt-chromium, cobalt-chromium alloys, PEEK, PEKK, carbon fiber reinforced PEEK, carbon fiber reinforced PEKK, or other suitable metal or non-metal biocompatible material. Of course, it is understood that the relative size of the components can be modified for the particular vertebra(e) to be instrumented and for the particular location or structure of the vertebrae to which the anchor assembly will be engaged.

It is contemplated but not required that the various interbody elements of differing sizes and add-on elements of differing sizes and configurations such as those disclosed herein can be provided in a kit of implant components. The surgeon can select and assemble the add-on element to the interbody element during surgery in order to provide the desired implant assembly based on conditions encountered during surgery.

Although various embodiments have been described as having particular features and/or combinations of components, other embodiments are possible having a combination of any features and/or components from any of embodiments as discussed above. As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the terms “proximal” and “distal” refer to the direction closer to and away from, respectively, an operator (e.g., surgeon, physician, nurse, technician, etc.) who would insert the medical implant and/or instruments into the patient. For example, the portion of a medical instrument first inserted inside the patient's body would be the distal portion, while the opposite portion of the medical device (e.g., the portion of the medical device closest to the operator) would be the proximal portion.

While the invention has been illustrated and described in detail in the drawings and foregoing description, the same is to be considered as illustrative and not restrictive in character, it being understood that all changes and modifications that come within the spirit of the invention are desired to be protected.

What is claimed is:

1. An implant assembly for spinal stabilization, comprising:
an interbody element including a body positionable in a space between first and second vertebrae, said interbody element extending from a leading end wall to an opposite trailing end, said body of said interbody element including a superior bone engaging surface and an opposite inferior bone engaging surface extending between said leading end wall and said trailing end, said superior and inferior bone engaging surfaces engageable with respective endplates of the first and second vertebrae when said interbody element is positioned between the first and second vertebrae; and
an add-on element attachable to said trailing end of said body of said implant element, said add-on element including an outer facing surface and an opposite inner facing surface oriented toward said trailing end of said interbody element, said add-on element further including a superior bone engaging surface for engaging the first vertebra and an inferior bone engaging surface for engaging the second vertebra when said add-on element is attached to said interbody element, wherein one of said trailing end of said interbody element and said inner surface of said add-on element includes a receiving portion having a groove that opens superiorly or inferiorly and the other of said interbody element and said add-on element includes a mating element positionable through said groove opening so that said add-on element is attachable to said interbody element by rotating said add-on element relative to said interbody element to move said mating element along said groove until said add-on element is aligned with said interbody element.

2. The implant assembly of claim 1, wherein said add-on element includes an elongated body extending between opposite sides that are aligned with opposite laterally facing surfaces of said interbody element when said add-on element is attached to said interbody element, said opposite sides of said add-on element and said laterally facing surfaces of said interbody element each include grooves that align with one another when said add-on element is mated to and aligned with said interbody element to receive fingers extending from an inserter instrument.

3. The implant assembly of claim 1, wherein said superior bone engaging surface and said inferior bone engaging surface of said add-on element each include a number of projections extending therefrom for engaging the endplate of a respective one of the first and second vertebrae.

4. The implant assembly of claim 1, wherein said mating element includes a cylindrical shape with an enlarged outer end and a recessed portion extending around said enlarged outer end and said groove includes an enlarged inner portion for receiving said enlarged outer end of said mating element and said groove further includes a reduced width portion for receiving said recessed portion of said mating element.

5. The implant assembly of claim 1, wherein:
said one of said trailing end of said interbody element and said inner surface of said add-on element includes first and second receiving portions located at opposite sides thereof and said groove of one of said receiving portions opens superiorly and a groove of the other of said receiving portions opens inferiorly; and
said other of said interbody element and said add-on element includes a pair of mating elements located at opposite sides thereof that are positionable through respective ones of said groove openings so that said add-on element is rotatable relative to said interbody element to move said mating elements along said grooves until add-on element is aligned with said interbody element.

6. The implant assembly of claim 5, wherein said interbody element includes first and second lateral walls extending from said leading end wall to said trailing end, said lateral walls being located on opposite sides of a cavity of said body of said interbody element.

7. The implant assembly of claim 6, wherein said first receiving portion is located in said trailing end at said first lateral wall and said second receiving portion is located in said trailing end at said second lateral wall.

8. The implant assembly of claim 1, wherein:
said add-on element includes a body defining a central plane alignable with the sagittal plane when positioned in the space between the first and second vertebrae, and said body of said add-on element is configured to be positioned substantially entirely in the space between the first and second vertebrae when said add-on element is engaged to and aligned with said interbody element; and
said body of said add-on element includes first and second apertures extending therethrough, said first aperture being configured for receiving a first bone anchor to engage the first vertebra and said second aperture being configured for receiving a second bone anchor to engage the second vertebra when said add-on element is engaged to and aligned with said interbody element.

9. The implant assembly of claim 8, wherein said first and second apertures through said body of said add-on element are angled toward said sagittal plane in a direction toward said interbody element.

10. The implant assembly of claim 8, wherein said add-on element includes a first set of teeth-like projections extending superiorly from a portion of said body of said add-on element around said first aperture for engaging an endplate of the first vertebra and said add-on element includes a second set of teeth-like projections extending inferiorly from a portion of said body of said add-on element around said second aperture for engaging an endplate of the second vertebra.

11. The implant assembly of claim 1, wherein:
said add-on element includes a body defining a central plane alignable with the sagittal plane when positioned in the space between the first and second vertebrae, and said body of said add-on element is configured to be positioned substantially entirely in the space between the first and second vertebrae when said add-on element is engaged to and aligned with said interbody element, said add-on element further including a first plate-like projection extending superiorly from said body of said add-on element and a second plate-like projection extending inferiorly from said body of said add-on element; and
said first and second projections of said add-on element each include an aperture extending therethrough, and said apertures are angled to extend superiorly and inferiorly away from one another in a direction toward said interbody element when said add-on element is engaged to and aligned with said interbody element, said first angled aperture being configured for receiving a first bone anchor to engage the first vertebra and said second angled aperture being configured for receiving a second bone anchor to engage the second vertebra.

12. The implant assembly of claim 11, wherein said apertures of said first and second projections are aligned with one another on said central plane.

13. The implant assembly of claim 11, wherein said apertures of said first and second projections are offset from one another on opposite sides of said central plane.

14. The implant assembly of claim 11, where said aperture of said first projection includes a first pair of apertures offset on opposite sides of said central plane and said aperture of said second projection includes a second pair of apertures offset on opposite sides of said central plane.

15. The implant assembly of claim 11, wherein said interbody element includes a trailing end surface that extends between opposite lateral surfaces of said body.

16. A method for assembly of an interbody spinal implant, comprising:
positioning an add-on element to a first orientation where a first mating element projecting from the add-on element is located adjacent to a superior opening of a first receiving portion in a trailing end of an interbody element and where a second mating element projecting from the add-on element is located adjacent to an inferior opening of a second receiving portion in the trailing end of the interbody element; and
rotating the add-on element to move the first and second mating elements through respective ones of the superior and inferior openings of the first and second receiving portions to secure the add-on element to the trailing end of the interbody element, wherein the interbody element and add-on element are configured to be positioned in a space between first and second vertebrae in engagement with endplates of the first and second vertebrae.

17. The method of claim 16, wherein:
the add-on element defines a central plane that bisects a space between the first and second mating elements and the interbody element defines a central plane that bisects the trailing end of the interbody element, and in the first orientation the central plane of the add-on element is obliquely oriented to the central plane of the interbody element; and
rotating the add-on element includes rotating the add-on element until the central plane of the add-on element is aligned with the central plane of the interbody element to secure the add-on element to the trailing end of the interbody element.

18. The method of claim 16, wherein the first and second mating elements each includes a cylindrical shape with an enlarged outer end and a recessed portion extending around the enlarged outer end and the first and second receiving portions each include a groove with an enlarged inner portion in the interbody element for receiving the enlarged outer end of the respective mating element and the groove includes a reduced width portion between the groove and the trailing end for receiving the recessed portion of the respective mating element.

19. The method of claim 16, wherein the first and second mating elements are located adjacent to opposite sides of the add-on element.

20. The method of claim 16, further comprising engaging an inserter along opposite sides of the add-on element and the interbody element, wherein the inserter includes opposite fingers that each extend into grooves of the add-on element and the interbody element that are aligned with one another on opposite sides of the add-on element and the interbody element.

21. The method of claim 20, wherein the add-on element includes at least one first aperture for receiving a first bone anchor for engaging a first vertebra and at least one second aperture for receiving a second bone anchor for engaging a second vertebra, and the inserter includes a guide body with first and second passages aligned with respective ones of the first and second apertures of the add-on element.

22. The method of claim 16, further comprising a reaming guide that includes a body positionable in the space between first and second vertebrae, the reaming guide further including first and second guide flanges extending superiorly and inferiorly, respectively, from the body, and a central shaft extending proximally from the body to a proximal handle, wherein the first and second guide flanges each include at least one passage to receive a reamer to form a notch in respective ones of the first and second vertebrae that receive superior and inferior projections extending from the add-on element in recessed relation to outer surfaces of the first and second vertebrae.
